(19) United States
(12) Patent Application Publication
(10) Pub. No.: US 2026/0021139 A1
(43) Pub. Date: Jan. 22, 2026

(54) COMBINATION OF SMALL MOLECULE DRUG CONJUGATE AND CAR-EXPRESSING CYTOTOXIC LYMPHOCYTES AND METHODS OF USE

(71) Applicant: Purdue Research Foundation, West Lafayette, IN (US)

(72) Inventors: Philip S. Low, West Lafayette, IN (US); Weichuan Luo, Rockville, MD (US)

(21) Appl. No.: 18/869,271

(22) PCT Filed: May 26, 2023
(86) PCT No.: PCT/US2023/067561

§ 371 Date: Nov. 25, 2024

Related U.S. Application Data
(60) Provisional application No. 63/346,152, filed on May 26, 2022.

(51) Int. Cl. A61K 35/17; A61K 40/11; A61K 40/15; A61K 40/31; A61K 40/42; A61K 47/54

(52) U.S. Cl. CPC A61K 35/17; A61K 40/11; A61K 40/15; A61K 40/31; A61K 40/4211; A61K 47/545

(57) ABSTRACT

A method of treating a cancer in a subject comprising administering to the subject (i) a small molecule drug conjugate (SMDC), which targets a cell-surface receptor on an immunosuppressive cell or a cancerous cell, and (ii) cytotoxic lymphocytes, which express a chimeric antigen receptor (CAR); and synergistic combinations of a SMDC and CAR-expressing cytotoxic lymphocytes.

### PRIORITY

This application is related to and claims the priority benefit of U.S. Provisional Patent Application No. 63/346,152 filed May 26, 2022. The content of the aforementioned application is hereby incorporated by reference in its entirety into this disclosure.

### TECHNICAL FIELD

The present disclosure includes combinations (e.g., synergistic combinations) of small molecule drug conjugates (or pharmaceutically acceptable salts thereof) and chimeric antigen receptor-expressing cytotoxic lymphocytes, as well as methods of treating cancer using such combinations and use of the same for the preparation of medicaments.

### SUMMARY

Provided is a method of treating a cancer in a subject, which method comprises administering to the subject (e.g., a therapeutically effective amount of):
a small molecule drug conjugate (SMDC) comprising:
   (a) a drug moiety (E) selected from the group consisting of a phosphoinositide 3-kinase (PI3K) inhibitor, a stimulator of interferon genes (STING) agonist, nucleotide-binding oligomerization domain (NOD)-like receptor (NLR) agonist, a retinoic acid-inducible gene-I (RIG-I)-like receptor (RLR) agonist, an absent in melanoma 2 (AIM2)-like receptor (ALR) agonist, a receptor for advanced glycation end products (RAGE) agonist, a kinase of the Pelle/interleukin-1 (IL-1) receptor-associated kinase 1 (IRAK1) agonist, an IRAK2 agonist, an IRAK4 agonist, an IRAK3 antagonist, an Src homology 2 domain-containing tyrosine phosphatase 1 and 2 (SHP1/2) antagonist, a T cell protein tyrosine phosphatase (TC-PTP) antagonist, a diacylglycerol kinase (DGK) antagonist, an enhancer of zeste homolog 2 (EZH2) antagonist, a transforming growth factor beta (TGFβ) antagonist, a nuclear factor kappa-light-chain-enhancer of activated B cells (NFκβ) activator, or a 1-kappa-β (Iκβ) kinase antagonist; and
   (b) a ligand (A) selected from the group consisting of a radical of raltitrexed, a radical of 5-methyltetrahydrofolate, and a group comprising formula (X)

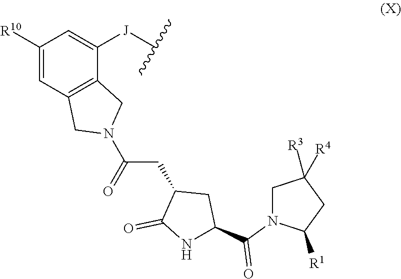

wherein:
   J is (C(R^{J})₂)₀₋₃, wherein each R^{J} is H, or two or more R^{J} are taken together to form oxo;
   R¹ is selected from the group consisting of —CN, —CHO, and —B(OH)₂;
   R³ and R⁴ are each independently selected from the group consisting of —H and F; and
   R¹⁰ is selected from the group consisting of H, —CF₃, F, Cl, Br, and I; and
      (c) a linker (L) connecting the ligand (A) and the drug moiety (E); and
   a chimeric antigen receptor (CAR)-expressing cytotoxic lymphocyte;
whereupon the subject is treated for cancer.

In some embodiments, the ligand comprises a radical of raltitrexed or a radical of 5-methyltetrahvdrofolate. In some embodiments, the ligand is a group comprising formula (X). In some embodiments, J is CH₂; in some embodiments, J is a bond (e.g., J is (C(R^{J})₂)₀). In some embodiments, the ligand (A) comprises formula (XA):

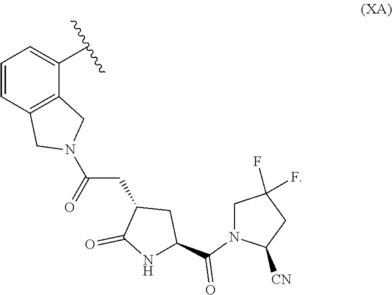

In some embodiments, the ligand (A) comprises formula (XB):

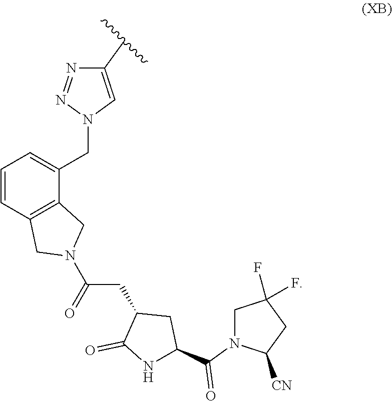 [SMILES: *c1cn(Cc2cccc3c2CN(C(=O)C[C@@H]2C[C@@H](C(=O)N4CC(F)(F)C[C@H]4C#N)NC2=O)C3)nn1]

In some embodiments, the ligand (A) comprises formula (XC):

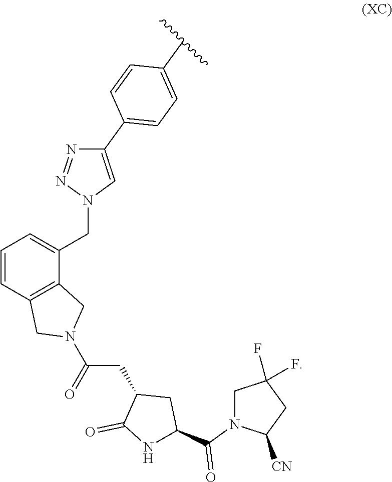 [SMILES: *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

In some embodiments, the ligand (A) comprises formula (XD):

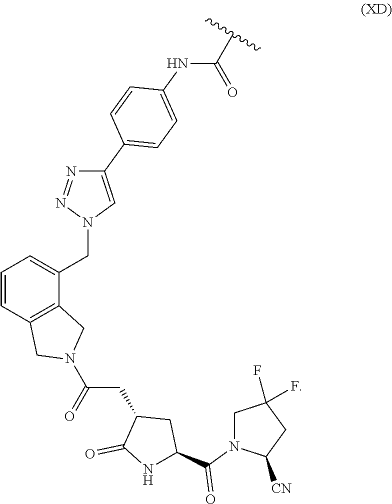 [SMILES: *C(=O)Nc1ccc(-c2cn(Cc3cccc4c3CN(C(=O)C[C@@H]3C[C@@H](C(=O)N5CC(F)(F)C[C@H]5C#N)NC3=O)C4)nn2)cc1]

In some embodiments, the ligand comprises formula (XA), formula (XB), formula (XC), or formula (XD):

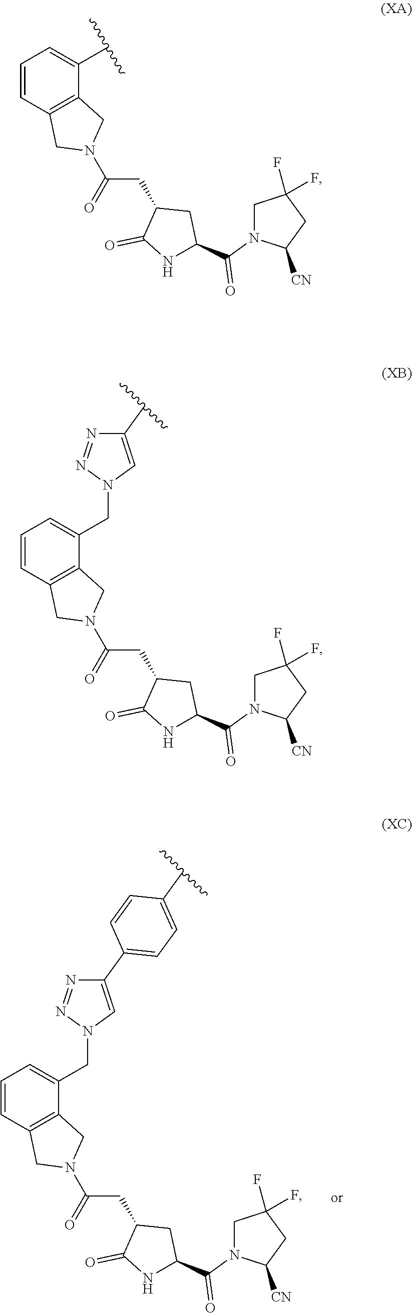 [SMILES: *c1cccc2c1CN(C(=O)C[C@@H]1C[C@@H](C(=O)N3CC(F)(F)C[C@H]3C#N)NC1=O)C2 ; *c1cn(Cc2cccc3c2CN(C(=O)C[C@@H]2C[C@@H](C(=O)N4CC(F)(F)C[C@H]4C#N)NC2=O)C3)nn1 ; *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

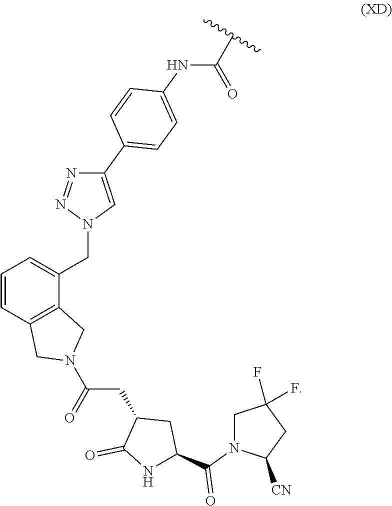 [SMILES: *C(=O)Nc1ccc(-c2cn(Cc3cccc4c3CN(C(=O)C[C@@H]3C[C@@H](C(=O)N5CC(F)(F)C[C@H]5C#N)NC3=O)C4)nn2)cc1]

In some embodiments, the drug moiety is a PI3K inhibitor, and the ligand is a radical of raltitrexed. In some embodiments, the drug moiety is a PI3K inhibitor, and the ligand is a radical of 5-methyltetrahydrofolate. In some embodiments, the drug moiety is a PI3K inhibitor, and the ligand is a group comprising formula (X). In some embodiments, the drug moiety is a NFκβ activator, and the ligand is a radical of raltitrexed. In some embodiments, the drug moiety is a NFκβ activator, and the ligand is a radical of 5-methyltetrahydrofolate. In some embodiments, the drug moiety is a NFκβ activator, and the ligand is a group comprising formula (X). In some embodiments, the drug moiety is an Iκβ kinase inhibitor, and the ligand is a radical of raltitrexed. In some embodiments, the drug moiety is an Iκβ kinase inhibitor, and the ligand is a radical of 5-methyltetrahvdrofolate. In some embodiments, the drug moiety is an Iκβ kinase inhibitor, and the ligand is a group comprising formula (X). In some embodiments, the drug moiety is a STING agonist, and the ligand is a radical of raltitrexed. In some embodiments, the drug moiety is a STING agonist, and the ligand is a radical of 5-methyltetrahydrofolate. In some embodiments, the drug moiety is a STING agonist, and the ligand is a group comprising formula (X). In some embodiments, the drug moiety is a NLR agonist, and the ligand is a radical of raltitrexed. In some embodiments, the drug moiety is an NLR agonist, and the ligand (A) is a radical of 5-methyltetrahydrofolate. In some embodiments, the drug moiety is an NLR agonist, and the ligand is a group comprising formula (X). In some embodiments, the drug moiety is an RLR agonist, and the ligand is a radical of raltitrexed. In some embodiments, the drug moiety is an RLR agonist, and the ligand is a radical of 5-methyltetrahydrofolate. In some embodiments, the drug moiety is an RLR agonist, and the ligand is a group comprising formula (X). In some embodiments, the drug moiety is an ALR agonist, and the ligand is a radical of raltitrexed. In some embodiments, the drug moiety is an ALR agonist, and the ligand is a radical of 5-methyltetrahydrofolate. In some embodiments, the drug moiety is an ALR agonist, and the ligand is a group comprising formula (X). In some embodiments, the drug moiety is a RAGE agonist, and the ligand is a radical of raltitrexed. In some embodiments, the drug moiety is a RAGE agonist, and the ligand is a radical of 5-methyltetrahydrofolate. In some embodiments, the drug moiety is a RAGE agonist, and the ligand is a group comprising formula (X). In some embodiments, the drug moiety is an IRAK1 agonist, an IRAK2 agonist, an IRAK4 agonist, or an IRAK3 antagonist, and the ligand is a radical of raltitrexed. In some embodiments, the drug moiety is an IRAK1 agonist, an IRAK2 agonist, an IRAK4 agonist, or an IRAK3 antagonist, and the ligand is a radical of 5-methyltetrahydrofolate. In some embodiments, the drug moiety is an IRAK1 agonist, an IRAK2 agonist, an IRAK4 agonist, or an IRAK3 antagonist, and the ligand is a group comprising formula (X). In some embodiments, the drug moiety is a SHP1/2 inhibitor, and the ligand is a radical of raltitrexed. In some embodiments, the drug moiety is a SHP1/2 inhibitor, and the ligand is a radical of 5-methyltetrahydrofolate. In some embodiments, the drug moiety is a SHP1/2 inhibitor, and the ligand is a group comprising formula (X). In some embodiments, the drug moiety is a TC-PTP inhibitor, and the ligand is a radical of raltitrexed. In some embodiments, the drug moiety is a TC-PTP inhibitor, and the ligand is a radical of 5-methyltetrahydrofolate. In some embodiments, the drug moiety is a TC-PTP inhibitor, and the ligand is a group comprising formula (X). In some embodiments, the drug moiety is a DGK inhibitor, and the ligand is a radical of raltitrexed. In some embodiments, the drug moiety is a DGK inhibitor, and the ligand is a radical of 5-methyltetrahydrofolate. In some embodiments, the drug moiety is a DGK inhibitor, and the ligand (A) is a group comprising formula (X). In some embodiments, the drug moiety is a TGFβ inhibitor, and the ligand is a radical of raltitrexed. In some embodiments, the drug moiety is a TGFβ inhibitor, and the ligand is a radical of 5-methyltetrahvdrofolate. In some embodiments, the drug moiety is a TGFβ inhibitor, and the ligand is a group comprising formula (X).

In some embodiments, the linker comprises a releasable form of polyethylene glycol (PEG), a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol.

In some embodiments, the linker is a releasable linker. In some embodiments, the linker is anon-releasable linker. In some embodiments, the linker comprises a group represented by the structure:

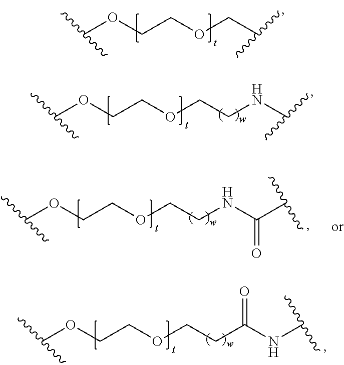

wherein w is 0-5 and t is 1-30. In some embodiments, the linker comprises one or more linker groups having the following structure:

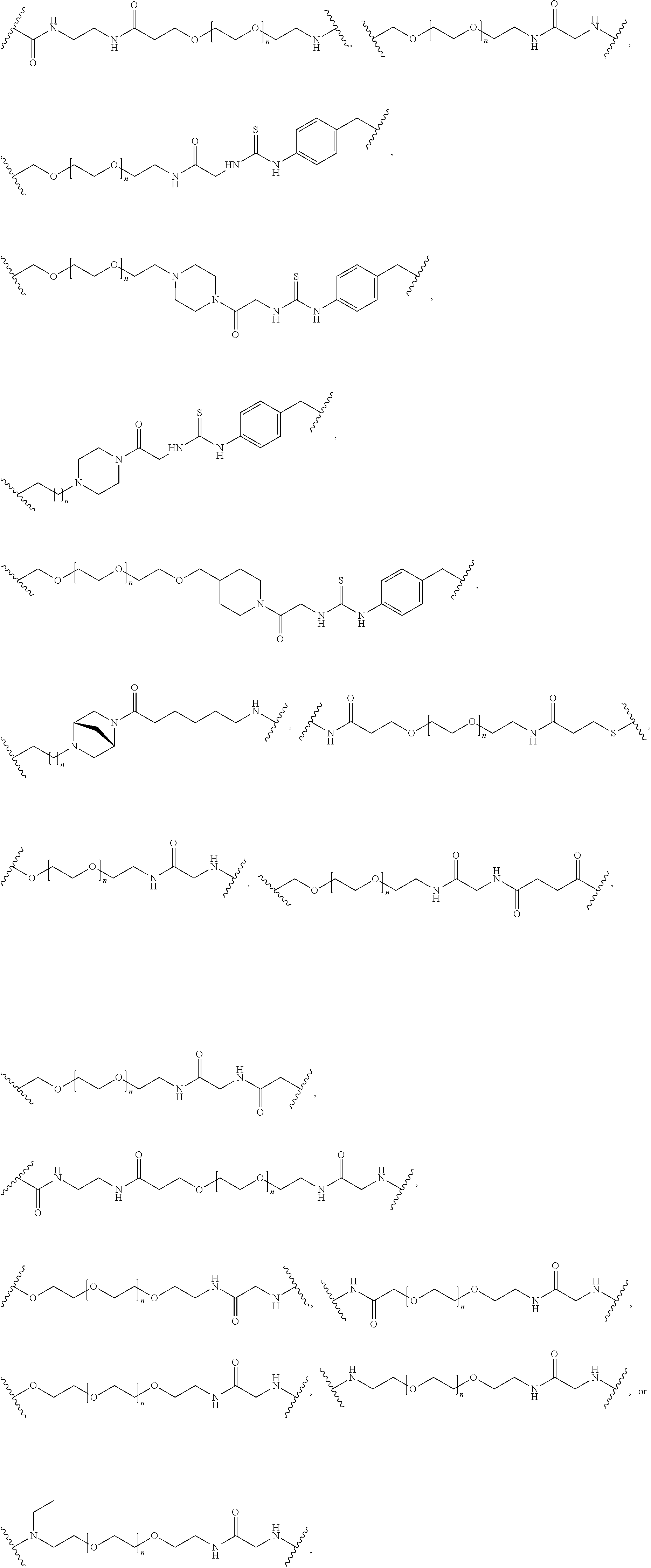

wherein n is 0 to 15. In some embodiments, the SMDC further comprises a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker. In some embodiments, the albumin binding group is selected from the group consisting of

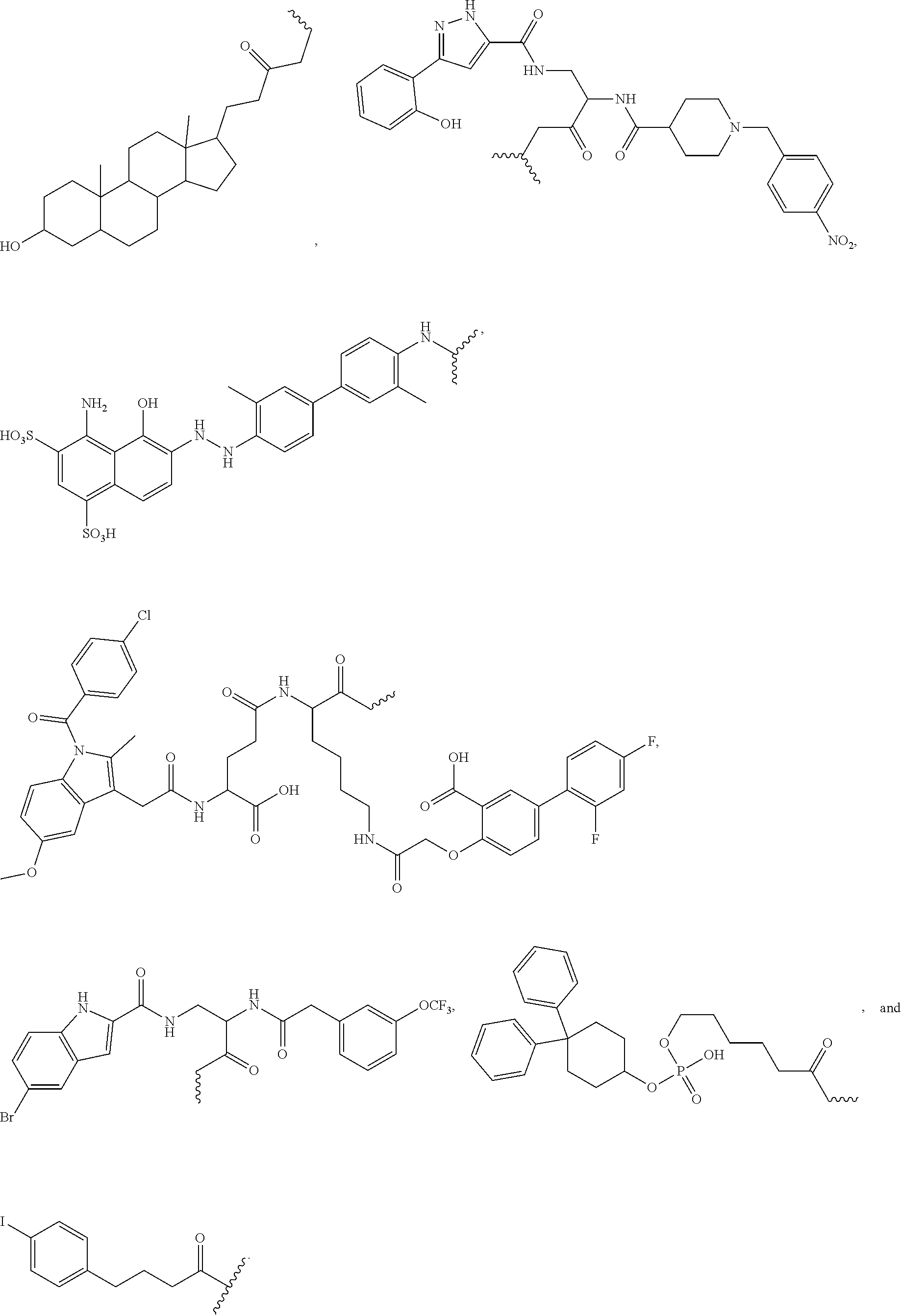

In some embodiments, each CAR comprises a recognition region, a co-stimulation domain, and an activation signaling domain. In some embodiments, the recognition region of the CAR is a single chain variable fragment (scFv) of an antibody that binds to a cell-surface antigen with high specificity. In some embodiments the CAR selectively binds a cell-surface antigen on an immunosuppressive cell or a cancer cell. In some embodiments, the CAR selectively binds a cell-surface antigen on an immunosuppressive cell or a cancer cell with specificity.

In some embodiments, the cell-surface antigen is a tumor-associated antigen (TAA). In some embodiments, the TAA is selected from the group consisting of CD19, CD20, CD30, CD3, CD4, CD5, BCMA, GD2, LEY, MS4A1, CD22, TNFRSFT7, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN. In some embodiments the TAA is CD19. In some embodiments, the CAR-expressing cytotoxic lymphocyte is a T cell, and the co-stimulation domain of the CAR is CD27, CD40L, CD70, 2B4, DNAM1, DAP12, DAP10, NKG2, NKG2D, CD28, CD137 (4-TBB), CD134 (OX40), or CD278 (ICOS).

In some embodiments, the CAR-expressing cytotoxic lymphocyte is a T cell, and the co-stimulation domain of the CAR is CD27, CD40L, CD70, 2B4, DNAM1, DAP12, DAP10, NKG2, NKG2D, CD28, CD137 (4-TBB), CD134 (OX40), or CD278 (ICOS). In some embodiments, the CAR-expressing cytotoxic lymphocyte is a NK cell, and the TAA is selected from the group consisting of CD3, CD4, and CD5. In some embodiments, the CAR-expressing cytotoxic lymphocyte is a NK cell, and the co-stimulation domain of the CAR is selected from the group consisting of 2B4, CD137 (4-1BB), DNAM1, DAP12, DAP10, NKG2, and NKG2D.

In some embodiments, the activation signaling domain of the CAR is a T cell CD3ζ chain or a Fc receptor γ. In some embodiments, the cytotoxic lymphocyte is autologous to the individual. In some embodiments, the cytotoxic lymphocyte is allogeneic. In some embodiments, the cytotoxic lymphocyte is heterologous to the individual. In some embodiments, the cancer is a solid tumor cancer. In some embodiments, the cancer (e.g., solid tumor cancer) is a cancer of the brain, thyroid, lung, pancreas, kidney, stomach, gastrointestinal stroma, endometrium, breast, cervix, ovary, colon, prostate, head, or neck. In some embodiments, the cancer is a leukemia, a lymphoma, or another was blood-related cancer. In some embodiments, the cancer is a folate receptor-expressing cancer or a cancer mediated by folate receptor-expressing immune cells. In some embodiments, the cancer is a folate receptor α-expressing cancer, a folate receptor β-expressing cancer, a folate receptor δ-expressing cancer, or a cancer mediated by folate receptor α, β, or δ-expressing immune cells. In some embodiments, the ligand is a radical of raltitrexed or a radical of 5-methyltetrahydrofolate and the cancer is a folate receptor-expressing cancer or a cancer mediated by folate receptor-expressing immune cells.

In some embodiments, the cancer is a cancer expressing fibroblast activation protein (FAP) or a cancer with cancer-associated fibroblasts expressing FAP. In some embodiments, the ligand is a group of formula (X), and the cancer is a cancer expressing FAP or a cancer with cancer associated fibroblasts which express FAP.

Administration of the therapeutically effective amount of the SMDC or a pharmaceutically acceptable salt thereof can inhibit cancer-associated fibroblast activity in the subject and increase the efficacy of the administered CAR-expressing cytotoxic lymphocytes against the cancer as compared to the efficacy of the CAR-expressing cytotoxic lymphocytes administered in the absence of the administration of a therapeutically effective amount of the SMDC or a pharmaceutically acceptable salt thereof.

Uses of a SMDC or a pharmaceutically acceptable salt thereof in the manufacture of a medicament for the treatment of cancer are also provided. In certain embodiments, the treatment comprises administering a therapeutically effective amount of the SMDC in combination with CAR T-cell therapy. The SMDC or pharmaceutically acceptable salt thereof can comprise any of the SMDCs hereof including, without limitation:
(a) a drug moiety selected from the group consisting of a PI3K inhibitor, a STING agonist, a NLR agonist, a RLR agonist, an ALR agonist, a RAGE agonist, an IRAK1 agonist, an IRAK2 agonist, an IRAK4 agonist, an IRAK3 antagonist, a SHP1/2 antagonist, a TC-PTP antagonist, a DGK antagonist, an EZH2 antagonist, a TGFβ antagonist, a NFκβ activator, or a Iκβ kinase antagonist;
(b) a ligand selected from the group consisting of a radical of raltitrexed, a radical of 5-methyltetrahvdrofolate, or a group comprising formula (X):

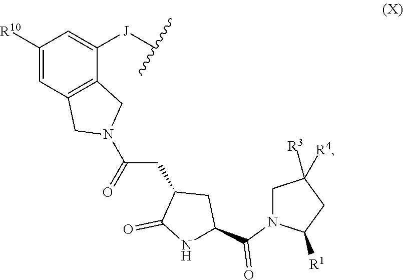

wherein:
   J is (C(R^{J})₂)₀₋₃, wherein each R^{J} is H, or two or more R^{J} are taken together to form oxo;
   R¹ is selected from the group consisting of —CN, —CHO, and —B(OH)₂;
   R³ and R⁴ are each independently selected from the group consisting of —H and F; and
   R¹⁰ is selected from the group consisting of H, —CF₃, F, Cl, Br and I; and
(c) a linker connecting the ligand and the drug moiety.

The ligand of the SMDC can comprise formula (XA), formula (XB), formula (XC), or formula (XD).

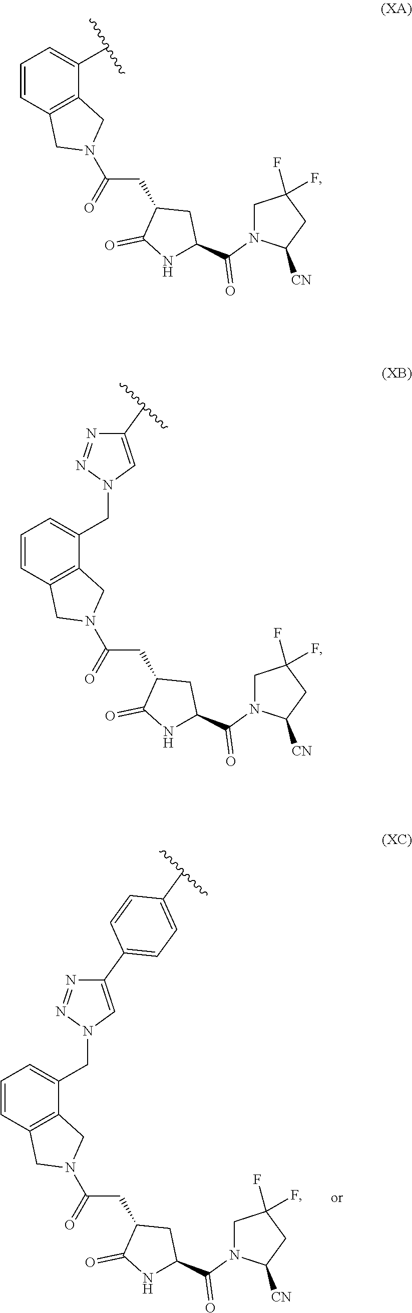 [SMILES: *c1cccc2c1CN(C(=O)C[C@@H]1C[C@@H](C(=O)N3CC(F)(F)C[C@H]3C#N)NC1=O)C2 ; *c1cn(Cc2cccc3c2CN(C(=O)C[C@@H]2C[C@@H](C(=O)N4CC(F)(F)C[C@H]4C#N)NC2=O)C3)nn1 ; *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

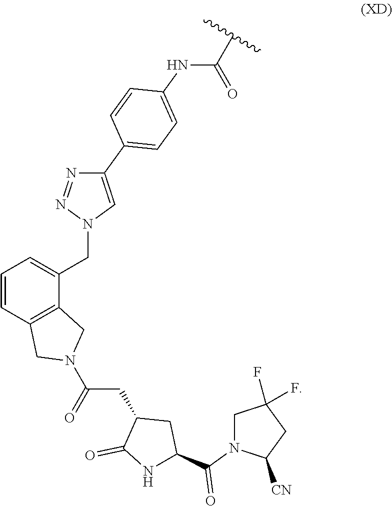 [SMILES: *C(=O)Nc1ccc(-c2cn(Cc3cccc4c3CN(C(=O)C[C@@H]3C[C@@H](C(=O)N5CC(F)(F)C[C@H]5C#N)NC3=O)C4)nn2)cc1]

The drug moiety can be selected from a group consisting of a PI3K inhibitor, NFκβ activator, an Iκβ kinase inhibitor, a STING agonist, a NLR agonist, a RLR agonist, an ALR agonist, a RAGE agonist, an agonist of IRAK1, an agonist of IRAK2, an agonist of IRAK4, an antagonist or inhibitor of IRAK3, a SHP1/2 inhibitor, a TC-PTP inhibitor, a DGK inhibitor, and a TGFβ inhibitor; and, in certain embodiments, the ligand can be a radical of raltitrexed, a radical of 5-methyltetrahydrofolate, or a group comprising formula (X). The linker can comprise a releasable form of PEG, a non-releasable form of PEG, polyproline, a hydrophilic amino acid, a sugar, an unnatural peptidoglycan, polyvinylpyrrolidone, a triblock copolymer comprising a central hydrophobic block of polypropylene glycol flanked on each side by a hydrophilic block of PEG, or a combination of any two or more of the foregoing. The linker can be a releasable linker. The linker can be a non-releasable linker. The linker can comprise a group represented by the structure:

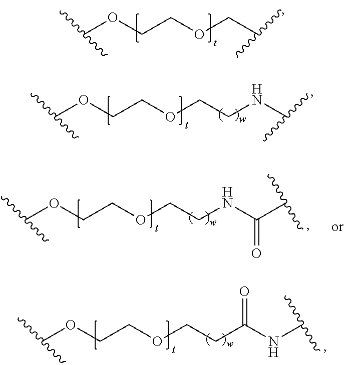

wherein w is 0-5 and t is 1-30. The linker can comprise one or more linker groups each independently having the follow structure:

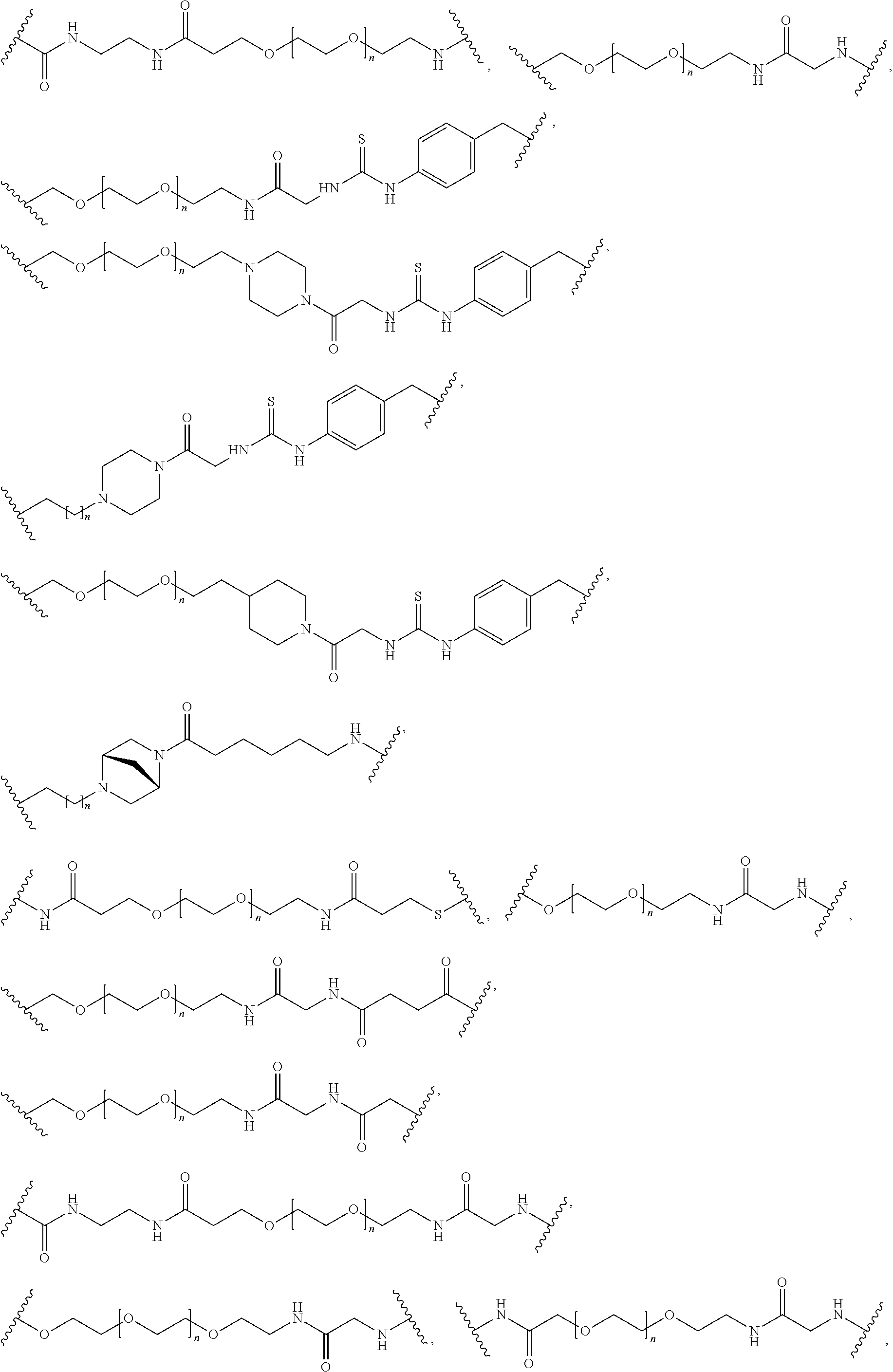

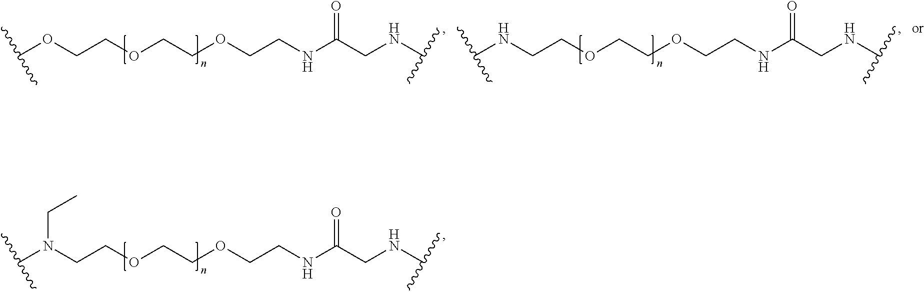

wherein n is 0 to 15.

In certain embodiments, the SMDC further comprises a radical of a PEG group, a peptide group, a glycopeptide group, a saccharide group, an albumnin-binding group or a radical of a combination of two or more of the foregoing groups, wherein the radical of the PEG group, the peptide group, the glycopeptide group, the saccharide group, the albumin-binding group, or the combination is attached to the linker. The CAR T-cell therapy can comprise administration of CAR-expressing cytotoxic lymphocytes to the subject, wherein each CAR comprises a recognition region, a co-stimulation domain, and an activation signaling domain.

The recognition region of the CAR can be a scFv of an antibody that binds to a cell-surface antigen with high specificity. The CAR can selectively bind a cell-surface antigen on an immunosuppressive cell or a cancer cell with specificity. The cell-surface antigen can be a TAA. The TAA can be selected from the group consisting of CD19, CD20, CD30, CD3, CD4, CD5, BCMA, GD2, LEY, MS4A1, CD22, TNFRSF17, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN. The TAA can be CD19.

The CAR-expressing cytotoxic lymphocyte can be a T cell and the co-stimulation domain of the CAR can be CD27, CD40L, CD70, 2B4, DNAM1, DAP12, DAP10, NKG2, NKG2D, CD28, CD137 (4-1BB), CD134 (OX40), or CD278 (ICOS). The CAR-expressing cytotoxic lymphocyte can be a NK cell and the TAA can be selected from the group consisting of CD3, CD4, and CD5.

The CAR-expressing cytotoxic lymphocyte can be a NK cell and the co-stimulation domain of the CAR can be selected from the group consisting of 2B4, CD137 (4-1BB), DNAM1, DAP12, DAP10, NKG2, and NKG2D. The activation signaling domain of the CAR can be a T cell CD3ζ chain or a Fc receptor γ. The cytotoxic lymphocyte can be autologous to the individual. The cytotoxic lymphocyte can be allogeneic. The cytotoxic lymphocyte can be heterologous to the individual. The cancer can be a solid tumor cancer. The solid tumor cancer can be a cancer of the brain, thyroid, lung, pancreas, kidney, stomach, gastrointestinal stroma, endometrium, breast, cervix, ovary, colon, prostate, head, or neck. The cancer can be a leukemia, a lymphoma, or another blood-related cancer. The ligand can be a radical of raltitrexed or a radical of 5-methyltetrahydrofolate and the cancer can be a folate receptor-expressing cancer or a cancer mediated by folate receptor-expressing immune cells. The cancer can be a folate receptor α-expressing cancer, a folate receptor β-expressing cancer, a folate receptor δ-expressing cancer, or a cancer mediated by folate receptor α, β, or δ-expressing immune cells. The ligand can be a group of formula (X), and the cancer is a cancer expressing FAP or a cancer with cancer associated fibroblasts which express FAP.

Synergistic combinations for treating a solid tumor cancer are also provided. In certain embodiments, a synergistic combination for treating a solid tumor cancer comprises: (i) a therapeutically effective amount of any SMDC described herein, or pharmaceutically acceptable salt thereof; and (ii) a therapeutically effective amount of a CAR-expressing cytotoxic lymphocyte. In certain embodiments, the SMDC of the synergist combination comprises:
(a) a drug moiety selected from the group consisting of a PI3K inhibitor, a STING agonist, a NLR agonist, a RLR agonist, an ALR agonist, a RAGE agonist, an IRAK1 agonist, an IRAK2 agonist, an IRAK4 agonist, an IRAK3 antagonist, a SHP1/2 antagonist, a TC-PTP antagonist, a DGK antagonist, an EZH2 antagonist, a TGFβ antagonist, a NFκβ activator, or a Iκβ kinase antagonist;
(b) a ligand selected from the group consisting of a radical of raltitrexed, a radical of 5-methyltetrahvdrofolate, or a group comprising formula (X):

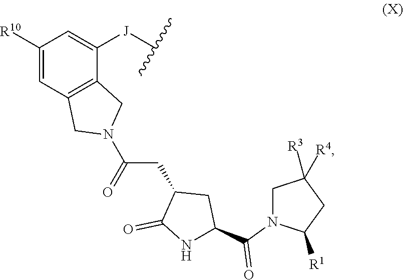

wherein:
   J is (C(R^{J})₂)₀₋₃, wherein each R^{J} is H, or two or more R^{J} are taken together to form oxo;
   R¹ is selected from the group consisting of —CN, —CHO, and —B(OH)₂;
   R³ and R⁴ are each independently selected from the group consisting of —H and F; and
   R¹⁰ is selected from the group consisting of H, —CF₃, F, Cl, Br and I; and
(c) a linker connecting the ligand and the drug moiety.

### DETAILED DESCRIPTION

The synergistic combinations and methods disclosed herein comprise a combination of (i) a small molecule drug conjugate (SMDC) or a pharmaceutically acceptable salt thereof comprising a drug moiety conjugated to a ligand, and (ii) chimeric antigen receptor (CAR)-expressing cytotoxic lymphocytes. Administration of the SMDC (or pharmaceutically acceptable salt thereof) is combined with the administration of CAR-expressing cytotoxic lymphocytes to augment the potency of the CAR cell therapy with little to no off-target toxicity observed.

The term “off-target toxicity” means organ or tissue damage, a reduction in the subject's weight that is not desirable to the physician or other individual treating the subject, or any other effect on the subject that is a potential adverse indicator to the treating physician (e.g., B cell aplasia, a fever, a drop in blood pressure, or pulmonary edema).

The terms “treat,” “treating,” “treated,” or “treatment” (with respect to a disease or condition) are used to describe an approach for obtaining beneficial or desired results, including preferably clinical results, which can include, but are not limited to, one or more of the following: improving a condition associated with a disease, curing a disease, lessening severity of a disease, increasing the quality of life of one suffering from a disease, prolonging survival, and/or a prophylactic or preventative treatment. In reference to cancer, in particular, the terms “treat,” “treating,” “treated,” or “treatment” can additionally mean reducing the size of a tumor, completely or partially removing the tumor (e.g., a complete or partial response), stabilizing disease, preventing progression of the cancer (e.g., progression-free survival), or any other effect on the cancer that would be considered by a physician to be a therapeutic, prophylactic, or preventative treatment of the cancer. None of the terms require the invention of a medical professional.

As used herein, “CAR therapy” and “CAR-T cells” refer to a cytotoxic lymphocyte cell (e.g., a T cell) or population thereof that has been modified through molecular biological methods to express a CAR on the cell surface. The CAR is a polypeptide having a pre-defined binding specificity to a desired target and is operably connected to (e.g., as a fusion, separate chains linked by one or more disulfide bonds, etc.) the intracellular part of a cell activation domain. By bypassing MHC class I and class II restriction, CAR engineered lymphocyte cells of both CD8+ and CD4+ subsets can be recruited for redirected target cell recognition.

“Binds with specificity,” “binds with high affinity,” or “specifically” or “selectively” binds, when referring to a ligand/receptor, a recognition region/targeting moiety, a nucleic acid/complementary nucleic acid, an antibody/antigen, or other binding pair indicates a binding reaction that is determinative of the presence of the protein in a heterogeneous population of proteins and other biologics. Thus, under designated conditions, a specified ligand or recognition region binds to a particular receptor (e.g., one present on a cancer cell) or targeting moiety, respectively, and does not bind in a significant amount to other proteins present in the sample (e.g., those associated with normal, healthy cells). Specific binding or binding with high affinity can also mean, for example, that the binding compound, ligand, antibody, or binding composition derived from the antigen-binding site of an antibody binds to its target with an affinity that is often at least 25% greater, more often at least 50% greater, most often at least 100% (2-fold) greater, normally at least ten times greater, more normally at least 20 times greater, and most normally at least 100 times greater than the affinity with any other binding compound.

In a typical embodiment, a molecule that specifically binds a target will have an affinity that is at least about 10⁶ liters/mol (K_{D}=10⁻⁶ M), and preferably at least about 10 liters/mol, as determined, for example, by Scatchard analysis. It is recognized by one of skill in the art that some binding compounds can specifically bind to more than one target, for example an antibody specifically binds to its antigen, to lectins by way of the antibody's oligosaccharide, and/or to an Fc receptor by way of the antibody's Fc region.

The administration of the SMDC or pharmaceutically acceptable salt thereof is combined with the administration of CAR-expressing cytotoxic lymphocytes, which prevents the inactivation of such CAR-expressing lymphocytes in the tumor microenvironment (TME) that has been observed with conventional approaches and/or inhibits cancer-associated fibroblasts in the TME to reduce or prevent the production of collagen thereby. In some embodiments, the SMDC or pharmaceutically acceptable salt thereof targets immunosuppressive cells (and/or cancerous cells) in the tumor and delivers the drug moiety to the targeted cells, thereby enhancing the infiltration and activities of the CAR-expressing cytotoxic lymphocytes within the TME while also avoiding systemic toxicity. Administration of a SMDC, along with the CAR-expressing cytotoxic lymphocytes, results in better cytotoxicity against cancer cells in solid tumors as compared to administration of CAR-expressing cytotoxic lymphocytes alone. As such, when used to treat a subject with cancer (e.g., a solid tumor cancer), the combinations hereof are synergistic in that administration of such combinations increases the cytotoxicity and/or efficacy of the treatment as compared to the cytotoxicity and/or efficacy of CAR-expressing cytotoxic lymphocytes administered in the absence of the administration of a therapeutically effective amount of the SMDC or a pharmaceutically acceptable salt thereof.

Provided herein, in certain embodiments, is a method of treating a cancer in a subject. The method comprises administering to the subject:
a SMDC or pharmaceutically acceptable salt thereof (e.g., a therapeutically effective amount of a SMDC or pharmaceutically acceptable salt thereof) comprising:
   (a) a drug moiety (E) selected from the group consisting of a phosphoinositide 3-kinase (PI3K) inhibitor, a stimulator of interferon genes (STING) agonist, nucleotide-binding oligomerization domain (NOD)-like receptor (NLR) agonist, a retinoic acid-inducible gene-I (RIG-I)-like receptor (RLR) agonist, an absent in melanoma 2 (AIM2)-like receptor (ALR) agonist, a receptor for advanced glycation end products (RAGE) agonist, a kinase of the Pelle/interleukin-1 (IL-1) receptor-associated kinase 1 (IRAK1) agonist, an IRAK2 agonist, an IRAK4 agonist, an IRAK3 antagonist, an Src homology 2 domain-containing tyrosine phosphatase 1 and 2 (SHP1/2) antagonist, a T cell protein tyrosine phosphatase (TC-PTP) antagonist, a diacylglycerol kinase (DGK) antagonist, an enhancer of zeste homolog 2 (EZH2) antagonist, a transforming growth factor beta (TGFβ) antagonist, a nuclear factor kappa-light-chain-enhancer of activated B cells (NFκβ) activator, or a 1-kappa-β (Iκβ) kinase antagonist; and
   (b) a ligand (A) selected from the group consisting of a radical of raltitrexed, a radical of 5-methyltetrahydrofolate, and a group comprising formula (X):

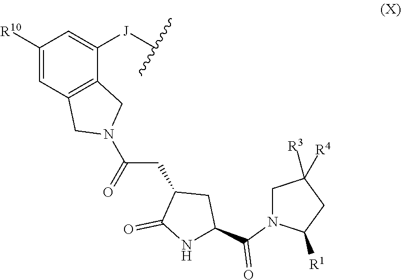

wherein:
   J is (C(R^{J})₂)₀₋₃, wherein each R^{J} is H, or two or more R^{J} are taken together to form oxo;
   R¹ is selected from the group consisting of —CN, —CHO, and —B(OH)₂;
   R³ and R⁴ are each independently selected from the group consisting of —H and F; and
   R¹⁰ is selected from the group consisting of H, —CF₃, F, Cl, Br and I; and
(c) a linker (L) connecting the ligand (A) and the drug moiety (E); and a CAR-expressing cytotoxic lymphocyte;
   whereupon the subject is treated for cancer.

### Small Molecule Drug Conjugates

In certain embodiments, the SMDCs for use in the combinations and methods hereof comprise (i) a drug moiety (E) selected from the group consisting of a PI3K inhibitor, a STING agonist, an NLR agonist, an RLR agonist, an ALR agonist, a RAGE agonist, an IRAK1 agonist, an IRAK2 agonist, an IRAK4 agonist, an IRAK3 antagonist, a SHP1/2 inhibitor, a TC-PTP inhibitor, a DGK inhibitor, an EZH2 inhibitor, a TGFβ inhibitor, a NFκβ activator, and an Iκβ kinase inhibitor; and (ii) a ligand (A) selected from the group consisting of a radical of raltitrexed, a radical of 5-methyltetrahydrofolate, and a group comprising formula (X):

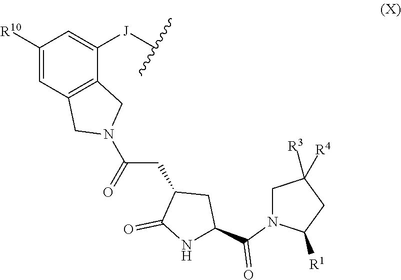

wherein:
   J is (C(R^{J})₂)₀₋₃, wherein each R^{J} is H, or two or more R^{J} are taken together to form oxo;
   R¹ is selected from the group consisting of —CN, —CHO, and —B(OH)₂;
   R³ and R⁴ are each independently selected from the group consisting of —H and F; and
   R¹⁰ is selected from the group consisting of H, —CF₃. F, Cl, Br and I; and
(iii) a linker (L) connecting the ligand (A) and drug moiety (E).

### Drug Moieties

In certain embodiments, SMDCs of the present disclosure comprise a drug moiety (E). Any drug useful for treating cancer can be used with an SMDC. In some embodiments, the drug moiety (E) is a STING agonist, a NLR agonist, a RLR agonist, an ALR agonist, a RAGE agonist, an IRAK1 agonist, an IRAK2 agonist, an IRAK4 agonist, an IRAK3 antagonist, a SHP1/2 antagonist, a TC-PTP antagonist, a DGK antagonist, an EZH2 antagonist, a TGFβ antagonist, a PI3K inhibitor, a NFκβ activator, or an Iκβ kinase antagonist. In some embodiments, the drug moiety (E) is a radical of a STING agonist, a NLR agonist, a RLR agonist, an ALR agonist, a RAGE agonist, an IRAK1 agonist, an IRAK2 agonist, an IRAK4 agonist, an IRAK3 antagonist, a SHP1/2 antagonist, a TC-PTP antagonist, a DGK antagonist, an EZH2 antagonist, a TGFβ antagonist, a PI3K inhibitor, a NFκβ activator, or an Iκβ kinase antagonist, e.g., a radical of a STING agonist, a NLR agonist, a RLR agonist, an ALR agonist, a RAGE agonist, an IRAK1 agonist, an IRAK2 agonist, an IRAK4 agonist, an IRAK3 antagonist, a SHP1/2 antagonist, a TC-PTP antagonist, a DGK antagonist, an EZH2 antagonist, a TGFβ antagonist, a PI3K inhibitor, a NFκβ activator, or an Iκβ kinase antagonist described herein.

The terms “activator” and “agonist” are used interchangeably herein and refer to a compound that interacts with a receptor and activates the receptor to produce a biological response. The terms “inhibitor” and “antagonist” are used interchangeably herein and refer to a compound that interacts with a receptor and blocks or reduces the receptor's ability to produce a biological response.

In some embodiments, the drug moiety (E) is a PI3K inhibitor.

In some embodiments, the drug moiety (E) is a NFκβ activator.

In some embodiments, the drug moiety (E) is an IKO kinase antagonist.

In some embodiments, the drug moiety (E) is a STING agonist.

In some embodiments, the drug moiety (E) is a NLR agonist.

In some embodiments, the drug moiety (E) is an RLR agonist.

In some embodiments, the drug moiety (E) is an ALR agonist.

In some embodiments, the drug moiety (E) is a RAGE agonist.

In some embodiments, the drug moiety (E) is an agonist of IRAK1. In some embodiments, the drug moiety (E) is an agonist of IRAK2. In some embodiments, the drug moiety (E) is an agonist of IRAK4. In some embodiments, the drug moiety (E) is an antagonist of IRAK3.

In some embodiments, the drug moiety (E) is a SHP1/2 antagonist.

In some embodiments, the drug moiety (E) is a TC-PTP antagonist.

In some embodiments, the drug moiety (E) is a DGK antagonist.

In some embodiments, the drug moiety (E) is an EZH2 antagonist.

In some embodiments, the drug moiety (E) is a TGFβ antagonist.

Exemplary NFκβ Activators are shown in Table 1.

TABLE 1
NFκβ Activators/Inducers

| Compound | Structure |
|---|---|
| AA | 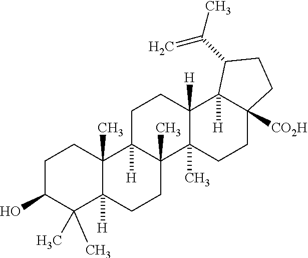 |
| BB | 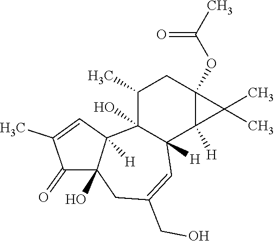 |
| CC | 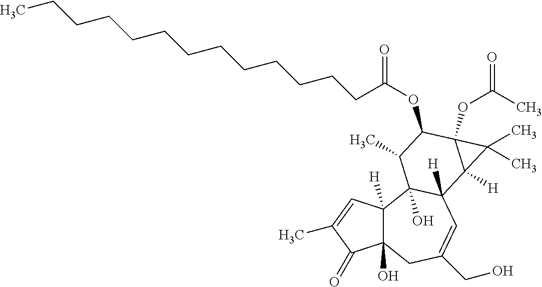 |
| DD | 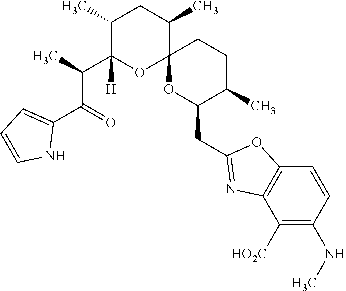 [SMILES: CNc1ccc2oc(C[C@H]3O[C@@]4(CC[C@H]3C)O[C@H]([C@H](C)C(=O)c3ccc[nH]3)[C@H](C)C[C@H]4C)nc2c1C(=O)O] |
| EE | 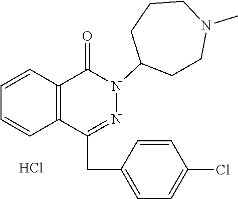 [SMILES: CN1CCCC(n2nc(Cc3ccc(Cl)cc3)c3ccccc3c2=O)CC1 ; Cl] |
| FF | 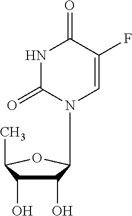 |
| GG | 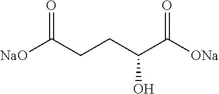 |

In some embodiments, the drug moiety (E) is a NFκβ activator, and the ligand (A) is (a radical of) raltitrexed. In some embodiments, the drug moiety (E) is a NFκβ activator, and the ligand (A) is (a radical of) 5-methyltetrahydrofolate. In some embodiments, the drug moiety (E) is a NFκβ activator, and the ligand (A) is a group comprising formula (X). In some embodiments, the drug moiety (E) is a NFκβ activator, and the ligand (A) is a group of or comprises formula (XA). In some embodiments, the drug moiety (E) is a NFκβ activator, and the ligand (A) is a group comprising formula (XB). In some embodiments, the drug moiety (E) is a NFκβ activator, and the ligand (A) is a group of or comprises formula (XC). In some embodiments, the drug moiety (E) is a NFκβ activator, and the ligand (A) is a group of or comprises formula (XD).

In some embodiments, the drug moiety (E) is a PI3K inhibitor. Non-limiting examples of PI3K inhibitors include, but are not limited to:

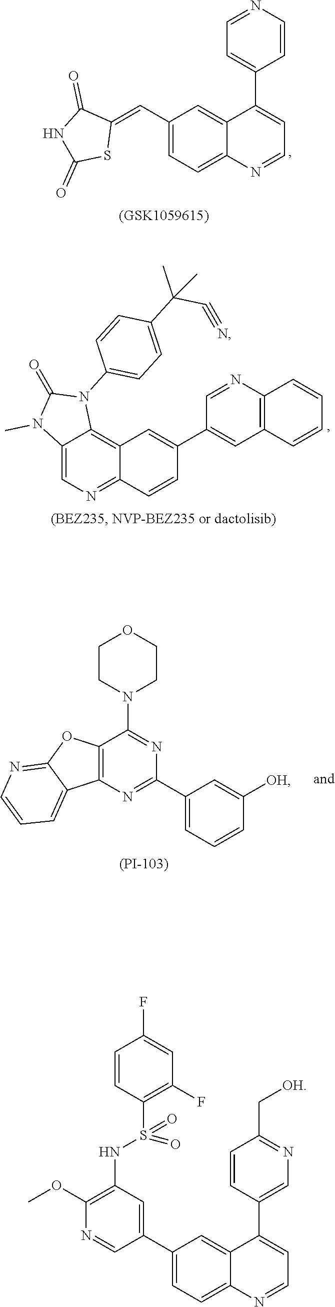 [SMILES: O=C1NC(=O)/C(=C/c2ccc3nccc(-c4ccncc4)c3c2)S1 ; Cn1c(=O)n(-c2ccc(C(C)(C)C#N)cc2)c2c3cc(-c4cnc5ccccc5c4)ccc3ncc21 ; Oc1cccc(-c2nc(N3CCOCC3)c3oc4ncccc4c3n2)c1 ; COc1ncc(-c2ccc3nccc(-c4ccc(CO)nc4)c3c2)cc1NS(=O)(=O)c1ccc(F)cc1F]

In some embodiments, the drug moiety (E) is a PI3K inhibitor, and the ligand (A) is (a radical of) raltitrexed. In some embodiments, the drug moiety (E) is a PI3K inhibitor, and the ligand (A) is (a radical of) 5-methyltetrahydrofolate. In some embodiments, the drug moiety (E) is a PI3K inhibitor, and the ligand (A) is a group comprising formula (X). In some embodiments, the drug moiety (E) is a PI3K inhibitor, and the ligand (A) is a group of or comprises formula (XA). In some embodiments, the drug moiety (E) is a PI3K inhibitor, and the ligand (A) is a group of or comprises formula (XB). In some embodiments, the drug moiety (E) is a PI3K inhibitor, and the ligand (A) is a group of or comprises formula (XC). In some embodiments, the drug moiety (E) is a PI3K inhibitor, and the ligand (A) is a group of or comprises formula (XD).

In some embodiments, the drug moiety (E) is a STING agonist.

Non-limiting examples of STING agonists include:

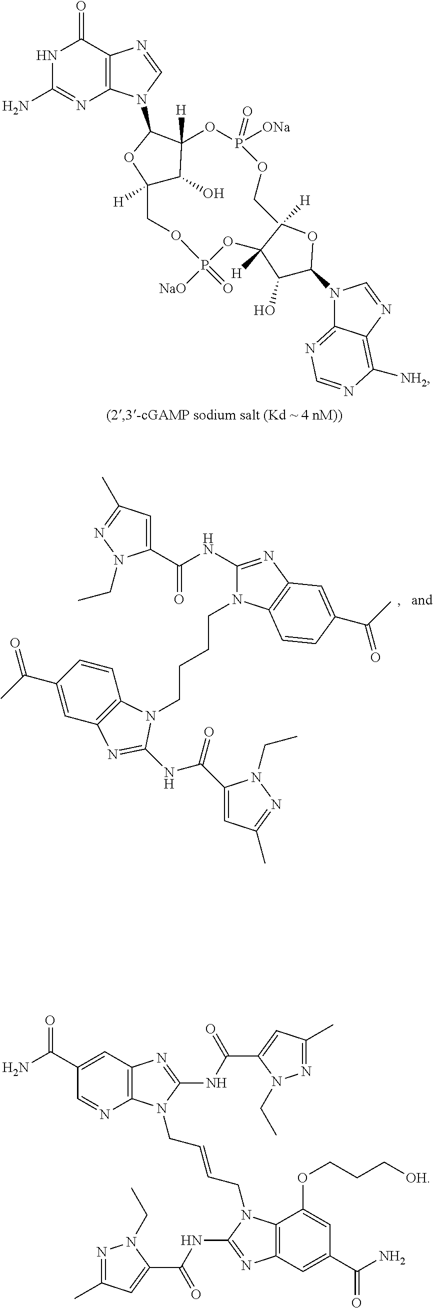

In some embodiments, the drug moiety (E) is a STING agonist, and the ligand (A) is (a radical of) raltitrexed. In some embodiments, the drug moiety (E) is a STING agonist, and the ligand (A) is (a radical of) 5-methyltetrahydrofolate. In some embodiments, the drug moiety (E) is a STING agonist, and the ligand (A) is a group comprising formula (X). In some embodiments, the drug moiety (E) is a STING agonist, and the ligand (A) is a group of or comprises formula (XA). In some embodiments, the drug moiety (E) is a STING agonist, and the ligand (A) is a group of or comprises formula (XB). In some embodiments, the drug moiety (E) is a STING agonist, and the ligand (A) is a group of or comprises formula (XC). In some embodiments, the drug moiety (E) is a STING agonist, and the ligand (A) is a group of or comprises formula (XD).

In some embodiments, the drug moiety (E) is an NLR agonist. A non-limiting example of an NLR agonist includes:

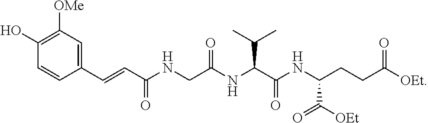 [SMILES: CCOC(=O)CC[C@@H](NC(=O)[C@@H](NC(=O)CNC(=O)/C=C/c1ccc(O)c(OC)c1)C(C)C)C(=O)OCC]

In some embodiments, the drug moiety (E) is a NLR agonist, and the ligand (A) is (a radical of) raltitrexed. In some embodiments, the drug moiety (E) is a NLR agonist, and the ligand (A) is (a radical of) 5-methyltetrahydrofolate. In some embodiments, the drug moiety (E) is a NLR agonist, and the ligand (A) is a group comprising formula (X). In some embodiments, the drug moiety (E) is a NLR agonist, and the ligand (A) is a group of or comprises formula (XA). In some embodiments, the drug moiety (E) is a NLR agonist, and the ligand (A) is a group of or comprises formula (XB). In some embodiments, the drug moiety (E) is a NLR agonist, and the ligand (A) is a group of or comprises formula (XC). In some embodiments, the drug moiety (E) is a NLR agonist, and the ligand (A) is a group of or comprises formula (XD).

In some embodiments, the drug moiety (E) is an Iκβ kinase inhibitor. In some embodiments, the drug moiety (E) is a Iκβ kinase inhibitor, and the ligand (A) is (a radical of) raltitrexed. In some embodiments, the drug moiety (E) is an Iκβ kinase inhibitor, and the ligand (A) is (a radical of) 5-methyltetrahydrofolate. In some embodiments, the drug moiety (E) is an Iκβ kinase inhibitor, and the ligand (A) is a group comprising formula (X). In some embodiments, the drug moiety (E) is an Iκβ kinase inhibitor, and the ligand (A) is a group of or comprises formula (XA). In some embodiments, the drug moiety (E) is an Iκβ kinase inhibitor, and the ligand (A) is a group of or comprises formula (XB). In some embodiments, the drug moiety (E) is an Iκβ kinase inhibitor, and the ligand (A) is a group of or comprises formula (XC). In some embodiments, the drug moiety (E) is an Iκβ kinase inhibitor, and the ligand (A) is a group of or comprises formula (XD).

In some embodiments, the drug moiety (E) is an RLR agonist. In some embodiments, the drug moiety (E) is an RLR agonist, and the ligand (A) is (a radical of) raltitrexed. In some embodiments, the drug moiety (E) is an RLR agonist, and the ligand (A) is (a radical of) 5-methyltetrahydrofolate. In some embodiments, the drug moiety (E) is an RLR agonist, and the ligand (A) is a group comprising formula (X). In some embodiments, the drug moiety (E) is an RLR agonist, and the ligand (A) is a group of or comprises formula (XA). In some embodiments, the drug moiety (E) is an RLR agonist, and the ligand (A) is a group of or comprises formula (XB). In some embodiments, the drug moiety (E) is an RLR agonist, and the ligand (A) is a group of or comprises formula (XC). In some embodiments, the drug moiety (E) is an RLR agonist, and the ligand (A) is a group of or comprises formula (XD).

In some embodiments, the drug moiety (E) is an ALR agonist. In some embodiments, the drug moiety (E) is an ALR agonist, and the ligand (A) is (a radical of) raltitrexed. In some embodiments, the drug moiety (E) is an ALR agonist, and the ligand (A) is (a radical of) 5-methyltetrahydrofolate. In some embodiments, the drug moiety (E) is an ALR agonist, and the ligand (A) is a group comprising formula (X). In some embodiments, the drug moiety (E) is an ALR agonist, and the ligand (A) is a group of or comprises formula (XA). In some embodiments, the drug moiety (E) is an ALR agonist, and the ligand (A) is a group of or comprises formula (XB). In some embodiments, the drug moiety (E) is an ALR agonist, and the ligand (A) is a group of or comprises formula (XC). In some embodiments, the drug moiety (E) is an ALR agonist, and the ligand (A) is a group of or comprises formula (XD).

In some embodiments, the drug moiety (E) is a RAGE agonist. In some embodiments, the drug moiety (E) is a RAGE agonist, and the ligand (A) is (a radical of) raltitrexed. In some embodiments, the drug moiety (E) is a RAGE agonist, and the ligand (A) is (a radical of) 5-methyltetrahydrofolate. In some embodiments, the drug moiety (E) is a RAGE agonist, and the ligand (A) is a group comprising formula (X). In some embodiments, the drug moiety (E) is a RAGE agonist, and the ligand (A) is a group of or comprises formula (XA). In some embodiments, the drug moiety (E) is a RAGE agonist, and the ligand (A) is a group of or comprises formula (XB). In some embodiments, the drug moiety (E) is a RAGE agonist, and the ligand (A) is a group of or comprises formula (XC). In some embodiments, the drug moiety (E) is a RAGE agonist, and the ligand (A) is a group of or comprises formula (XD).

In some embodiments, the drug moiety (E) is an agonist of IRAK1, an agonist of IRAK2, an agonist of IRAK4, or an antagonist of IRAK3. In some embodiments, the drug moiety (E) is an agonist of IRAK1, an agonist of IRAK2, an agonist of IRAK4, or an antagonist of IRAK3, and the ligand (A) is (a radical of) raltitrexed. In some embodiments, the drug moiety (E) is an agonist of IRAK1, an agonist of IRAK2, an agonist of IRAK4, or an antagonist of IRAK3, and the ligand (A) is (a radical of) 5-methyltetrahydrofolate. In some embodiments the drug moiety (E) is an agonist of IRAK1, an agonist of IRAK2, an agonist of IRAK4, or an antagonist of IRAK3, and the ligand (A) is a group comprising formula (X). In some embodiments the drug moiety (E) is an agonist of IRAK1, an agonist of IRAK2, an agonist of IRAK4, or an antagonist of IRAK3, and the ligand (A) is a group of or comprises formula (XA). In some embodiments the drug moiety (E) is an agonist of IRAK1, an agonist of IRAK2, an agonist of IRAK4, or an antagonist of IRAK3, and the ligand (A) is a group of or comprises formula (XB). In some embodiments the drug moiety (E) is an agonist of IRAK1, an agonist of IRAK2, an agonist of IRAK4, or an antagonist of IRAK3, and the ligand (A) is a group of or comprises formula (XC). In some embodiments the drug moiety (E) is an agonist of IRAK1, an agonist of IRAK2, an agonist of IRAK4, or an antagonist of IRAK3, and the ligand (A) is a group of or comprises formula (XD).

In some embodiments, the drug moiety (E) is a SHP1/2 inhibitor. In some embodiments, the drug moiety (E) is a SHP1/2 inhibitor, and the ligand (A) is (a radical of) raltitrexed. In some embodiments, the drug moiety (E) is a SHP1/2 inhibitor, and the ligand (A) is (a radical of) 5-methyltetrahydrofolate. In some embodiments, the drug moiety (E) is a SHP1/2 inhibitor, and the ligand (A) is a group comprising formula (X). In some embodiments, the drug moiety (E) is a SHP1/2 inhibitor, and the ligand (A) is a group of or comprises formula (XA). In some embodiments, the drug moiety (E) is a SHP1/2 inhibitor, and the ligand (A) is a group of or comprises formula (XB). In some embodiments, the drug moiety (E) is a SHP1/2 inhibitor, and the ligand (A) is a group of or comprises formula (XC). In some embodiments, the drug moiety (E) is a SHP1/2 inhibitor, and the ligand (A) is a group of or comprises formula (XD).

In some embodiments, the drug moiety (E) is a TC-PTP inhibitor. In some embodiments, the drug moiety (E) is a TC-PTP inhibitor, and the ligand (A) is (a radical of) raltitrexed. In some embodiments, the drug moiety (E) is a TC-PTP inhibitor, and the ligand (A) is (a radical of) 5-methyltetrahydrofolate. In some embodiments, the drug moiety (E) is a TC-PTP inhibitor, and the ligand (A) is a group comprising formula (X). In some embodiments, the drug moiety (E) is a TC-PTP inhibitor, and the ligand (A) is a group of or comprises formula (XA). In some embodiments, the drug moiety (E) is a TC-PTP inhibitor, and the ligand (A) is a group of or comprises formula (XB). In some embodiments, the drug moiety (E) is a TC-PTP inhibitor, and the ligand (A) is a group of or comprises formula (XC). In some embodiments, the drug moiety (E) is a TC-PTP inhibitor, and the ligand (A) is a group of or comprises formula (XD).

In some embodiments, the drug moiety (E) is a DGK inhibitor. In some embodiments, the drug moiety (E) is a DGK inhibitor, and the ligand (A) is (a radical of) raltitrexed. In some embodiments, the drug moiety (E) is a DGK inhibitor, and the ligand (A) is (a radical of) 5-methyltetrahydrofolate. In some embodiments, the drug moiety (E) is a DGK inhibitor, and the ligand (A) is a group comprising formula (X). In some embodiments, the drug moiety (E) is a DGK inhibitor, and the ligand (A) is a group of or comprises formula (XA). In some embodiments, the drug moiety (E) is a DGK inhibitor, and the ligand (A) is a group of or comprises formula (XB). In some embodiments, the drug moiety (E) is a DGK inhibitor, and the ligand (A) is a group of or comprises formula (XC). In some embodiments, the drug moiety (E) is a DGK inhibitor, and the ligand (A) is a group of or comprises formula (XD).

In some embodiments, the drug moiety (E) is an EZH2 inhibitor. Non-limiting examples of EZH2 inhibitors include:

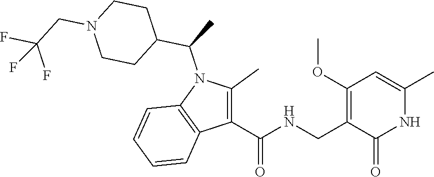 [SMILES: COc1cc(C)[nH]c(=O)c1CNC(=O)c1c(C)n([C@H](C)C2CCN(CC(F)(F)F)CC2)c2ccccc12]

and tazemetostat. In some embodiments, the drug moiety (E) is an EZH2 inhibitor, and the ligand (A) is (a radical of) raltitrexed. In some embodiments, the drug moiety (E) is an EZH2 inhibitor, and the ligand (A) is (a radical of) 5-methyltetrahydrofolate. In some embodiments, the drug moiety (E) is an EZH2 inhibitor, and the ligand (A) is a group comprising formula (X). In some embodiments, the drug moiety (E) is an EZH2 inhibitor, and the ligand (A) is a group of or comprises formula (XA). In some embodiments, the drug moiety (E) is an EZH2 inhibitor, and the ligand (A) is a group of or comprises formula (XB). In some embodiments, the drug moiety (E) is an EZH2 inhibitor, and the ligand (A) is a group of or comprises formula (XC). In some embodiments, the drug moiety (E) is an EZH2 inhibitor, and the ligand (A) is a group of or comprises formula (XD).

In some embodiments, the drug moiety (E) is a TGFβ inhibitor. In some embodiments, the drug moiety (E) is a TGFβ inhibitor, and the ligand (A) is (a radical of) raltitrexed. In some embodiments, the drug moiety (E) is a TGFβ inhibitor, and the ligand (A) is (a radical of) 5-methyltetrahvdrofolate. In some embodiments, the drug moiety (E) is a TGFβ inhibitor, and the ligand (A) is a group comprising formula (X). In some embodiments, the drug moiety (E) is a TGFβ inhibitor, and the ligand (A) is a group of or comprises formula (XA). In some embodiments, the drug moiety (E) is a TGFβ inhibitor, and the ligand (A) is a group of or comprises formula (XB). In some embodiments, the drug moiety (E) is a TGFβ inhibitor, and the ligand (A) is a group of or comprises formula (XC). In some embodiments, the drug moiety (E) is a TGFβ inhibitor, and the ligand (A) is a group of or comprises formula (XD).

### Ligand/Targeting Moiety

The drug moiety of the SMDC is conjugated to a ligand (A) (e.g., a targeting moiety). A “drug moiety” as used herein is a radical of a molecule that is a drug or has drug-like properties. The term “radical” means a chemical group with an open valence for forming bonds with other atoms or molecules. A radical of a molecule can be formed by the removal of one or more atoms to leave an open valence. Unless otherwise stated, a radical is formed from a molecule by removal of a hydrogen atom.

In certain embodiments, the ligand is coupled with the drug moiety (E) via the linker (L). In some embodiments, the ligand (A) is selected from the group consisting of a radical of raltitrexed, a radical of 5-methyltetrahydrofolate, or a group comprising formula (X):

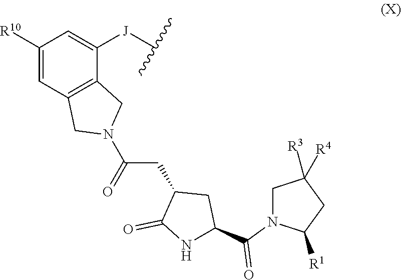

wherein:
   J is (C(R^{J})₂)₀₋₃, wherein each R^{J} is H, or two or more R^{J} are taken together to form oxo;
   R¹ is selected from the group consisting of —CN, —CHO, and —B(OH)₂;
   R³ and R⁴ are each independently selected from the group consisting of —H and F; and
   R¹⁰ is selected from the group consisting of H, —CF₃, F, Cl, Br and I.

In some embodiments, the ligand (A) comprises a radical of raltitrexed or a radical of 5-methyltetrahydrofolate. Raltitrexed has the structure:

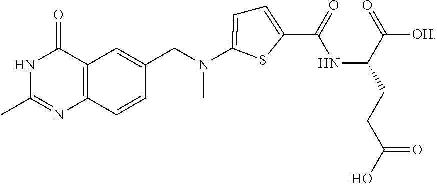

In some embodiments the ligand (A) has the structure:

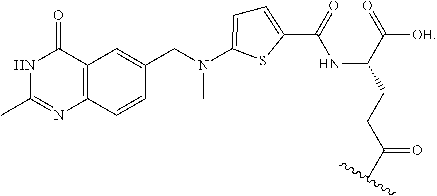 [SMILES: *C(=O)CC[C@H](NC(=O)c1ccc(N(C)Cc2ccc3nc(C)[nH]c(=O)c3c2)s1)C(=O)O]

5-methyltetrahydrofolate has the structure:

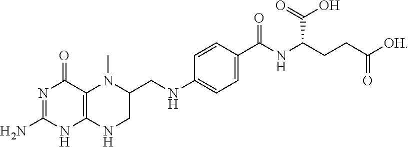 [SMILES: CN1c2c([nH]c(N)nc2=O)NCC1CNc1ccc(C(=O)N[C@@H](CCC(=O)O)C(=O)O)cc1]

In some embodiments the ligand (A) has the structure:

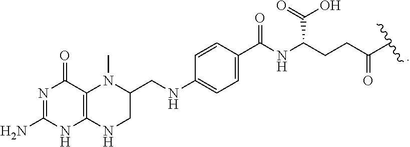 [SMILES: *C(=O)CC[C@H](NC(=O)c1ccc(NCC2CNc3[nH]c(N)nc(=O)c3N2C)cc1)C(=O)O]

In some embodiments, ligand (A) is a group comprising formula (X):

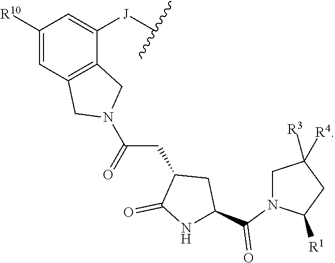

In some embodiments, J of formula (X) is CH₂. In some embodiments, J of formula (X) is C(O). In some embodiments, J of formula (X) is a bond (i.e., J is (C(R^{J})₂)₀). In some embodiments, R¹ of formula (X) is CN. In some embodiments, R¹⁰ of formula (X) is H. In some embodiments, R³ and R⁴ of formula (X) are each F. In some embodiments, R³ of formula (X) is H, and R⁴ of formula (X) is F.

In some embodiments, the ligand (A) is or comprises formula (XA):

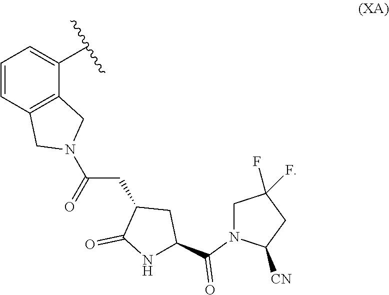 [SMILES: *c1cccc2c1CN(C(=O)C[C@@H]1C[C@@H](C(=O)N3CC(F)(F)C[C@H]3C#N)NC1=O)C2]

In some embodiments, the ligand (A) is or comprises formula (XB):

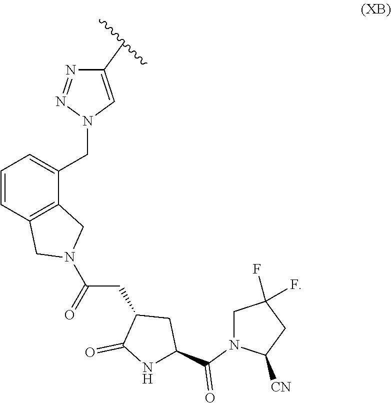 [SMILES: *c1cn(Cc2cccc3c2CN(C(=O)C[C@@H]2C[C@@H](C(=O)N4CC(F)(F)C[C@H]4C#N)NC2=O)C3)nn1]

In some embodiments, the ligand (A) is or comprises formula (XC):

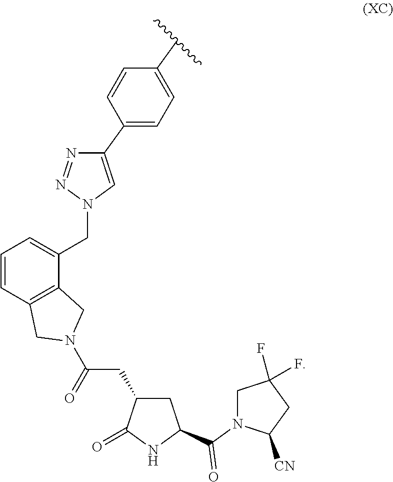 [SMILES: *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

In some embodiments, the ligand (A) is or comprises formula (XD):

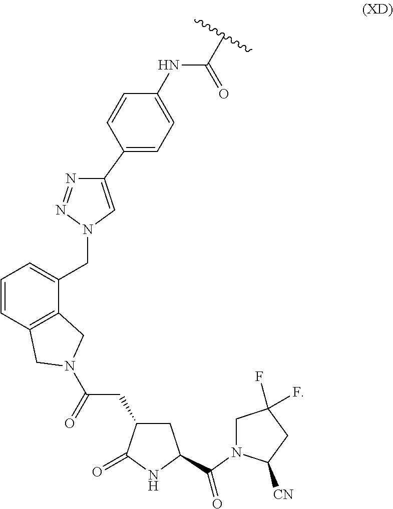 [SMILES: *C(=O)Nc1ccc(-c2cn(Cc3cccc4c3CN(C(=O)C[C@@H]3C[C@@H](C(=O)N5CC(F)(F)C[C@H]5C#N)NC3=O)C4)nn2)cc1]

In some embodiments, the ligand (A) comprises:

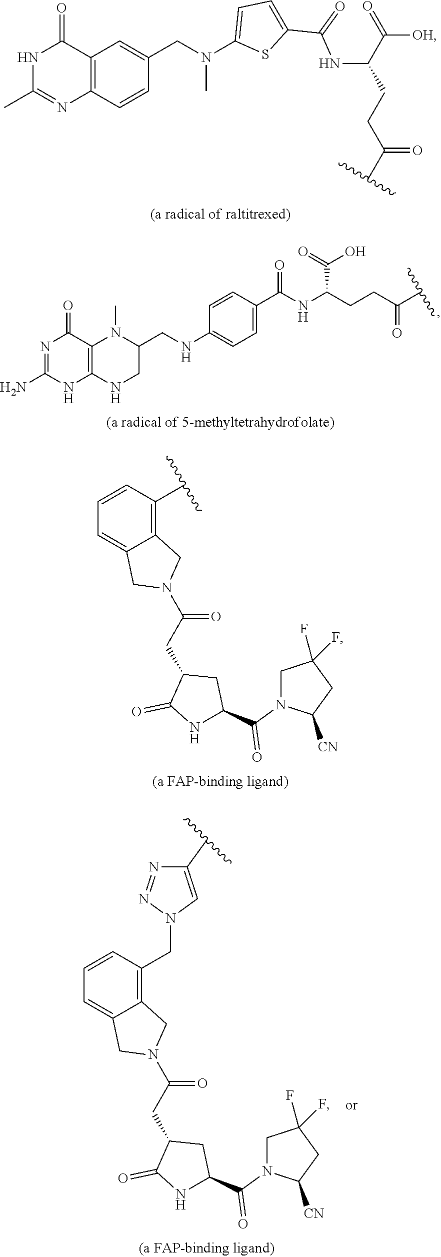 [SMILES: *C(=O)CC[C@H](NC(=O)c1ccc(N(C)Cc2ccc3nc(C)[nH]c(=O)c3c2)s1)C(=O)O ; *C(=O)CC[C@H](NC(=O)c1ccc(NCC2CNc3[nH]c(N)nc(=O)c3N2C)cc1)C(=O)O ; *c1cccc2c1CN(C(=O)C[C@@H]1C[C@@H](C(=O)N3CC(F)(F)C[C@H]3C#N)NC1=O)C2 ; *c1cn(Cc2cccc3c2CN(C(=O)C[C@@H]2C[C@@H](C(=O)N4CC(F)(F)C[C@H]4C#N)NC2=O)C3)nn1]

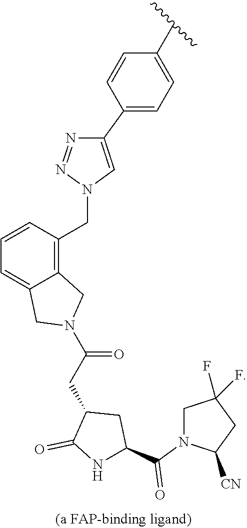 [SMILES: *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

### Likers

In some embodiments, the drug moiety and the ligand are conjugated via a linker (L). “Conjugate” and “compound” may be used interchangeably herein. More specifically, a “conjugate” is a kind of compound formed by the joining of two or more smaller chemical compounds. The term “linker” includes a chain of atoms that is bio-functionally adapted to form a chemical bond and connects a drug moiety and a ligand to form a conjugate. Illustratively, the chain of atoms can include carbon, nitrogen, oxygen, sulfur, silicon (Si), and phosphorus (P), such as C, N, O, S, and P, or C, N, O, and S. The linker can comprise a wide variety of links, such as in the range from about 2 to about 100 atoms in the contiguous backbone and can be releasable or non-releasable.

The linker can comprise a releasable form of polyethylene glycol (PEG), a non-releasable form of PEG, polyproline, a hydrophilic amino acid, a sugar, an unnatural peptidoglycan, polyvinylpyrrolidone, a triblock copolymer comprising a central hydrophobic block of polypropylene glycol flanked on each side by a hydrophilic block of polyethylene glycol, or a combination of any two or more of the foregoing. The linker can be (PEG)₃.

The term “releasable” in the context of a linker means a linker that includes at least one bond that can be broken (e.g., chemically or enzymatically hydrolyzed) under physiological conditions, such as, for example, by being reducing agent-labile, pH-labile, acid-labile, base-labile, oxidatively labile, metabolically labile, biochemically labile, or enzyme-labile or by being a p-aminobenzylic-based, multivalent, releasable bond. It is appreciated that the physiological conditions resulting in bond breaking do not necessarily include a biological or metabolic process and instead may include a standard chemical reaction, such as a hydrolysis reaction for example, at physiological pH or as a result of compartmentalization into a cellular organelle, such as an endosome having a lower pH than cytosolic pH. A cleavable bond can connect two adjacent atoms within the releasable linker and/or connect other linker portions or the targeting moiety/ligand and/or the drug, for example, at either or both ends of the releasable linker. In some instances, the releasable linker is broken into two or more fragments. In some instances, the releasable linker is separated from the targeting moiety/ligand. In some embodiments, the targeting moiety/ligand and the drug are released from each other, and the drug becomes active.

The term “non-releasable” in the context of a linker means a linker that includes at least one bond that is not easily or quickly broken under physiological conditions. A non-releasable linker can comprise a backbone that is stable under physiological conditions (e.g., the backbone is not susceptible to hydrolysis (e.g., aqueous hydrolysis or enzymatic hydrolysis)). A conjugate with a non-releasable linker does not release any component of the conjugate (e.g., a targeting ligand (e.g., a fully amorphous (FA)-ligand) or a drug (e.g., a PI3K inhibitor)). The non-releasable linker can lack a disulfide bond (e.g., S—S) or an ester in the backbone. The conjugate can comprise a targeting moiety/ligand and a drug connected by a backbone that is substantially stable for the entire duration of the conjugate's circulation (e.g., during endocytosis into the target cell endosome). A conjugate comprising a non-releasable linker can be particularly beneficial when the drug targets NOD-like receptors and/or other pattern recognition receptors present within the endosome of a cell. A non-releasable linker can comprise an amide, ester, ether, amine, and/or thioether (e.g., thio-maleimide). While specific examples are provided herein, it will be understood that any molecule(s) may be used in the non-releasable linker provided that at least one bond that is not easily or quickly broken under physiological conditions is formed.

A non-releasable linker comprises a linker that, at a neutral pH, for example, less than ten percent (10%) (e.g., less than 5%, less than 4%, less than 3%, less than 2%, less than 1%, less than 0.1%, less than 0.01%, or less than 0.001%) of which will hydrolyze in an aqueous (e.g., buffered (e.g., phosphate buffer) solution) within a period of time (e.g., 24 hours). Where a non-releasable linker is employed, less than about ten percent (10%), and preferably less than five percent (5%) or none, of the administered conjugate can release the free drug (e.g., in systemic circulation prior to uptake by the targeted cells/tissue). For example, within one (1) hour of administration, less than five percent (5%) of the free drug can be released from the conjugate while the compound is in systemic circulation. This can be beneficial as it can reduce off-target toxicity of the free drug.

Both releasable and non-releasable linkers can be engineered to optimize biodistribution, bioavailability, and PK/PD (e.g., of the compound) and/or to increase uptake (e.g., of the conjugate or drug) into the targeted tissue pursuant to methodologies commonly known in the art or hereinafter developed, such as through PEGylation and the like. In some embodiments the linker (L) is configured to avoid significant release of a pharmaceutically active amount of the drug in circulation prior to capture by a cell (e.g., a cell of interest (e.g., a macrophage in a tissue to be treated)).

Conjugates comprising releasable linkers can be designed to diffuse across the membrane of the endosome and, for example, into the cytoplasm of a targeted cell. Releasable linkers can be designed such that the drug is not released until the conjugate reaches the cytoplasm.

In some embodiments ligand (A) is attached to linker (L) of the compounds via a nitrogen atom (e.g., of the linker (L)). Ligand (A) can be attached to the linker (L) via a triazolyl or an amide (e.g., of the linker (L)).

In some embodiments the linker (L) is a releasable linker. In some embodiments the linker (L) is a non-releasable linker. In some embodiments the linker (L) comprises an optionally substituted heteroalkyl. The optionally substituted heteroalkyl is substituted with at least one substituent selected from the group consisting of alkyl, hydroxyl, acyl, polyethylene glycol (PEG), carboxylate, and halo. In some embodiments the linker (L) comprises a substituted heteroalkyl with at least one disulfide bond in the backbone thereof. In some embodiments the linker (L) comprises a peptide or a peptidoglycan with at least one disulfide bond in the backbone thereof. In some embodiments the linker (L) is a releasable linker that can be cleaved by enzymatic reaction, a reactive oxygen species (ROS), or reductive conditions. In some embodiments the linker (L) comprises the formula —NH—CH₂—CR⁶⁰R⁷⁰—S—S—CH₂—CH₂—O—CO—, wherein R⁶⁰ and R⁷⁰ are each, independently, H, alkyl, or heteroalkyl.

In some embodiments the linker (L) is a group or comprises a group of the formula:

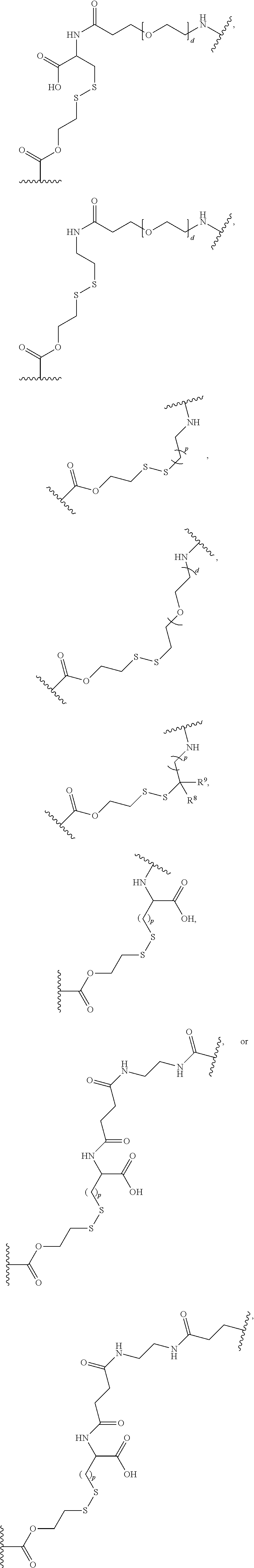

wherein p is an integer from 0 to 30; d is an integer from 1 to 40; and R⁸ and R⁹ are each, independently, H, alkyl, a heterocyclyl, a cycloalkyl, an aryl, or a heteroalkyl. In some embodiments the linker (L) comprises one or more linker moieties independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide, and peptidoglycan. In some embodiments the linker (L) is or comprises a peptide or a peptidoglycan.

In some embodiments the linker (L) is or comprises an amino acid. In some embodiments the linker (L) is or comprises a PEG group. In some embodiments the linker (L) is or comprises a polysaccharide. In some embodiments the linker (L) is or comprises a group represented by the structure:

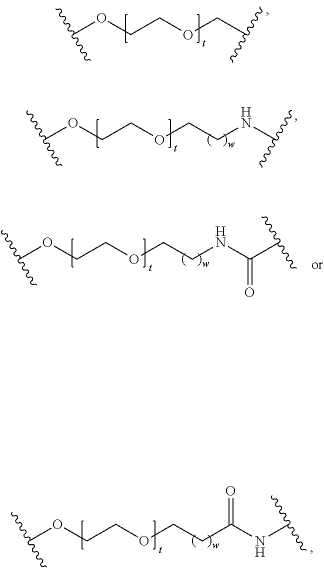

wherein w is 0-5 and t is 1-30.

In some embodiments the linker (L) is or comprises

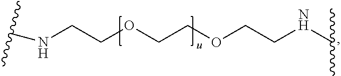

wherein u is an integer from 1 to 32. In some embodiments the linker (L) is or comprises

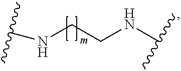

wherein m is an integer from 1 to 9. In some embodiments the linker (L) is or comprise

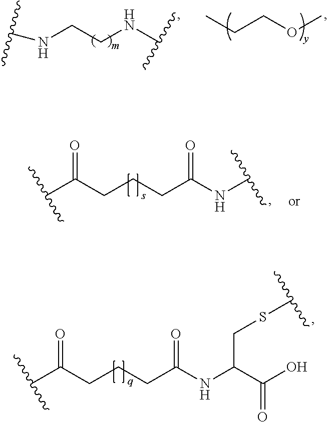

wherein m is an integer from 1 to 9, y is an integer from 1 to 32, q is an integer from 0 to 4; and s is an integer from 0 to 4.

In some embodiments the linker (L) comprises one or more linker groups having the following structure:

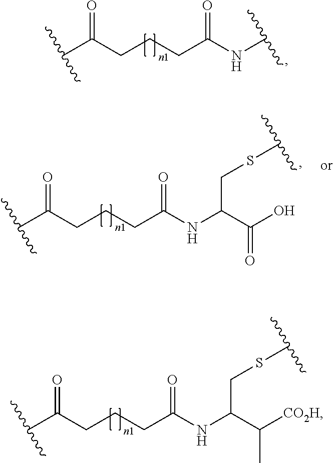

wherein n1 is 0 to 10.

In some embodiments the linker (L) comprises one or more linker groups independently having the following structure:

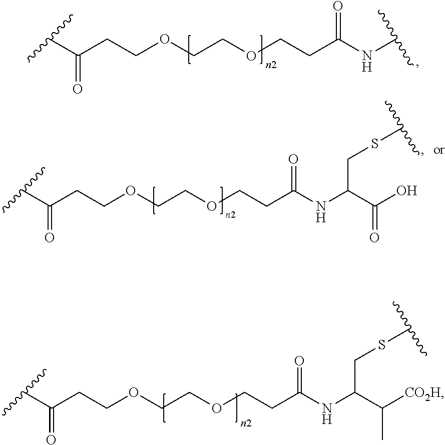

wherein n2 is 1 to 32.

In some embodiments the linker (L) comprises one or more linker groups having the following structure:

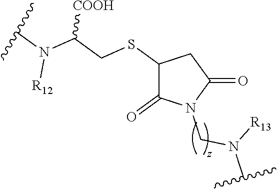

wherein:
R₁₂ and R₁₃ can each be independently H or C₁-C₆ alkyl; and
z is an integer from 1 to 8.

In some embodiments the linker (L) comprises one or more linker groups having the following structure:

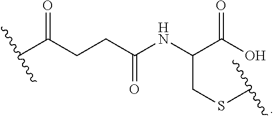 [SMILES: *SCC(NC(=O)CCC(*)=O)C(=O)O]

In some embodiments the linker (L) comprises one or more linker groups having the following structure:

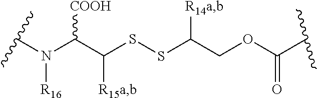

wherein: R₁₆ is H or C₁-C₆ alkyl; and R₁₄ₐ, R_{14b}, R₁₅ₐ, and R_{15b} can each be independently H or C₁-C₆ alkyl.

In some embodiments the linker (L) comprises the following structure:

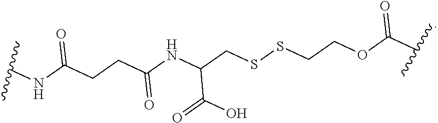 [SMILES: *NC(=O)CCC(=O)NC(CSSCCOC(*)=O)C(=O)O]

In some embodiments, the linker (L) comprises a releasable form of polyethylene glycol (PEG), a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol.

In some embodiments, the linker (L) is a releasable linker.

In some embodiments, the linker (L) is a non-releasable linker.

In some embodiments, the linker (L) comprises one or more linker groups having the following structure:

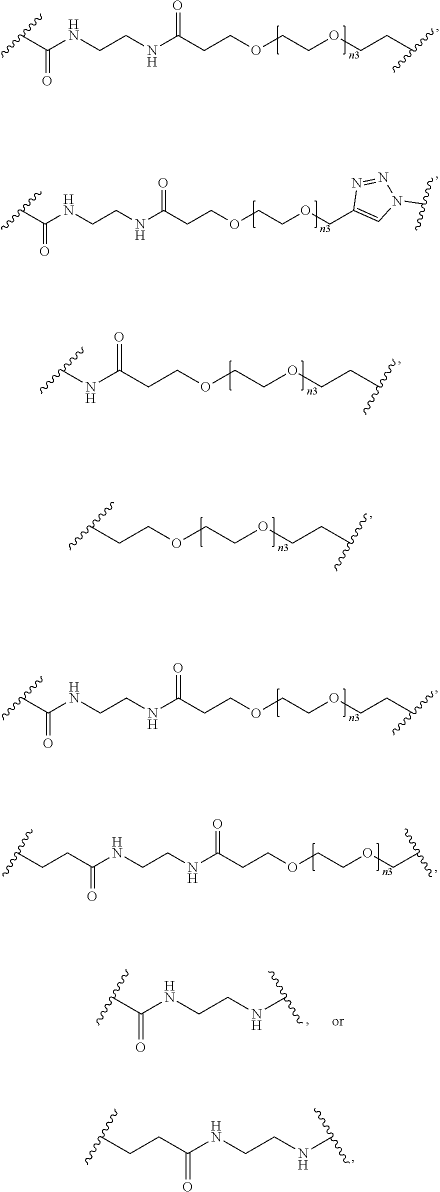

wherein n3 is 0 to 15.

In some embodiments, the linker (L) comprises one or more linker groups having the following structure:

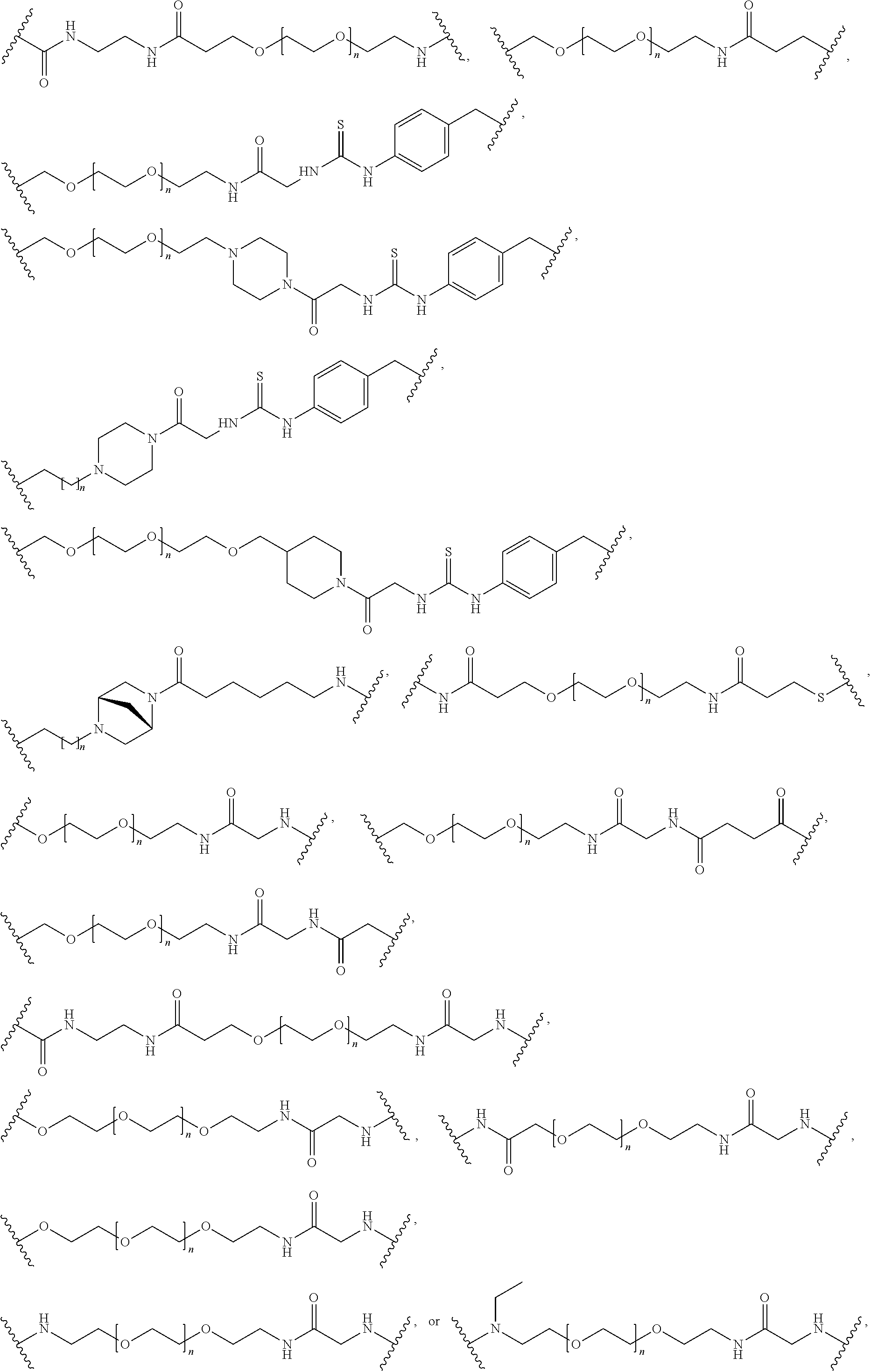

wherein n is 0 to 15.

In some embodiments the linker (L) is a substituted heteroalkyl comprising at least one substituent selected from the group consisting of alkyl, hydroxyl, oxo, PEG, carboxylate, and halo. “Halo” or “halogen,” by itself or as part of another substituent, means, unless otherwise stated, a fluorine, chlorine, bromine, or iodine atom.

In some embodiments, the linker (L) comprises a spacer (e.g., as described elsewhere herein). In some embodiments the spacer comprises a peptidoglycan or a sugar, for example. In some embodiments, the linker (L) is a substituted heteroalkyl with at least one disulfide bond in the backbone thereof. In some embodiments, the linker (L) is a peptide with at least one disulfide bond in the backbone thereof. In some embodiments, the linker (L) comprises —CONH—CH(COOH)—CH₂—S—S—CH₂—CRₐR_{b}—O—CO—, —CONH—CH(COOH)CRₐR_{b}—O—CO—, —C(O)NHCH(COOH)(CH₂)₂—CONH—CH(COOH)CRₐR_{b}—O—CO— or —C(O)NHCH(COOH)(CH₂)₂—CONH—CH(COOH)—CH₂—S—S—CH₂—CRₐR_{b}—O—CO—, wherein Rₐ and R_{b} are independently an H, an alkyl, or a heteroalkyl (e.g., PEG).

In some embodiments, the linker (L) comprises a structure of:

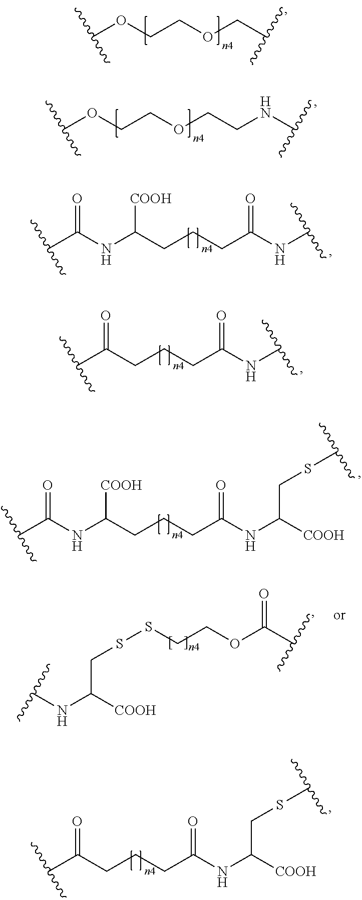

wherein n4 is 0 to 10.

In some embodiments, the linker (L) comprises a structure of

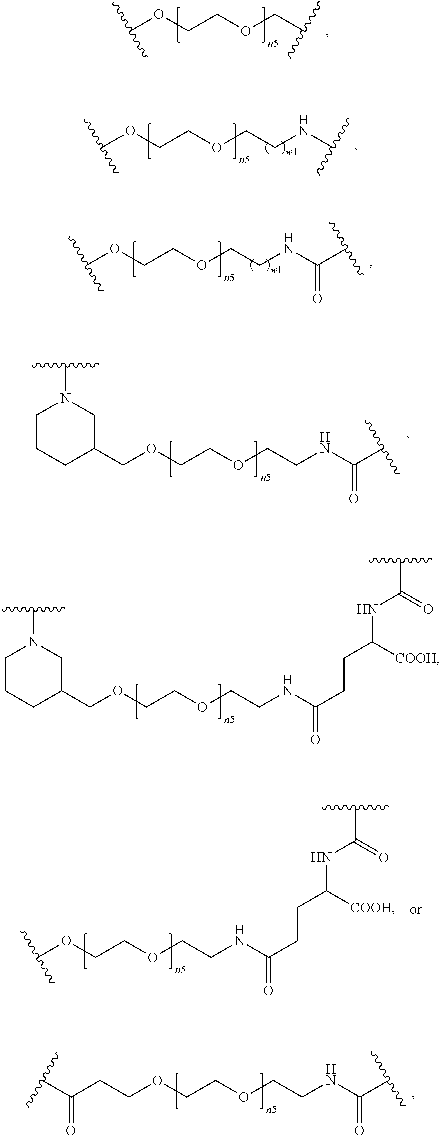

wherein n5 is 1 to 32 and w1 is 0-10. In at least one exemplary embodiment, n5 is 1 to 30 and w1 is 0 to 5 (where applicable).

In some embodiments, the linker (L) comprises the structure of

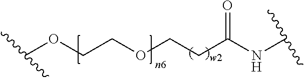

wherein n6 is 1 to 30 and w2 is 0 to 5.

In some embodiments, the linker (L) comprises a releasable form of PEG, a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol.

In some embodiments, the SMDC further comprises a radical of a PEG group, a peptide group, a glycopeptide group, a saccharide group, an albumin-binding group, or a radical of a combination of two or more of the foregoing groups, wherein the radical of the PEG group, the peptide group, the glycopeptide group, the saccharide group, the albumin-binding group, or the combination is attached to the linker (L).

In some embodiments, the albumin binding group is selected from the group consisting of

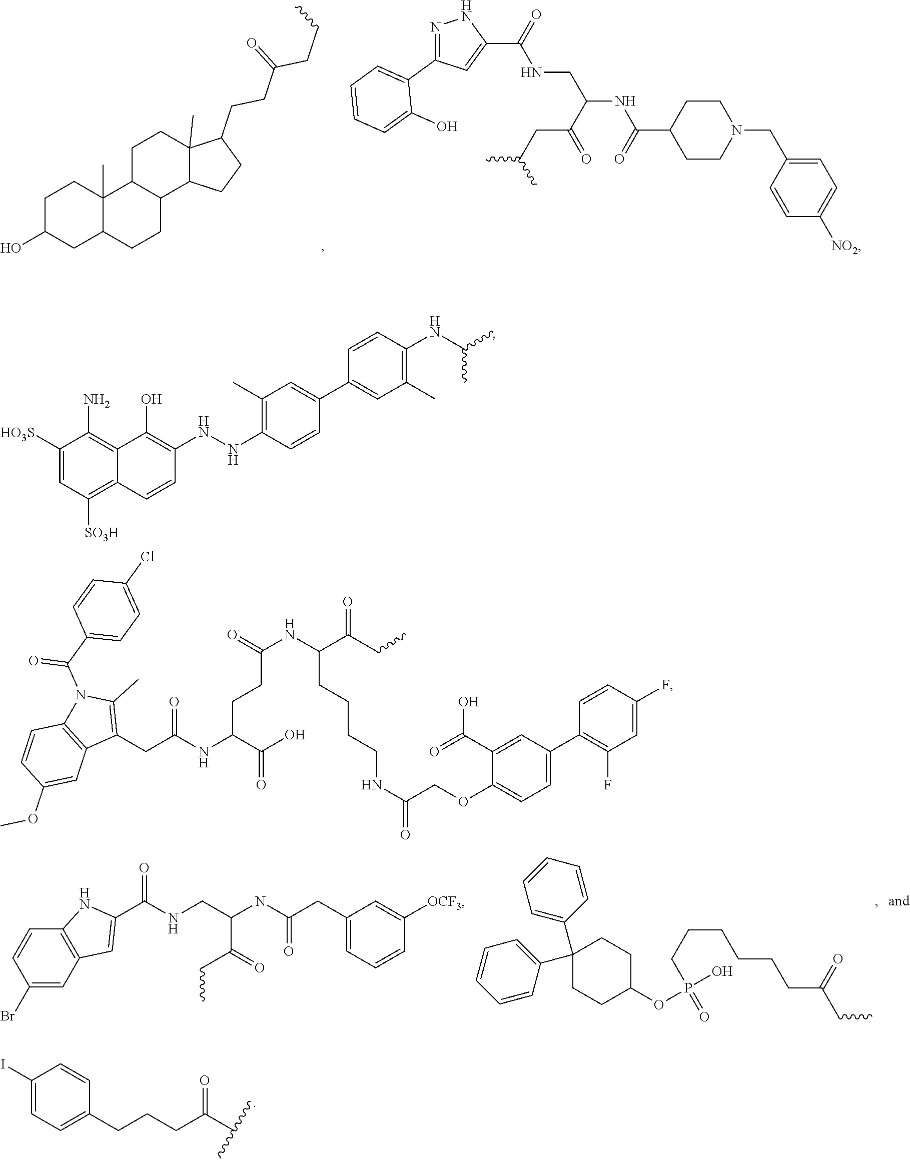

### Compounds and Administration

More than one SMDC can be administered and, in some instances, the SMDCs can comprise different drugs. One or more SMDCs can be administered in a composition along with one or more conjugated and/or unconjugated drugs. The SMDCs (e.g., drug moieties) can be used in accordance with the methods described herein and, in some instances, depending on the desired application, can be combined with other drugs that deplete or inhibit myeloid-derived suppressor cells (e.g., in connection with treatment for cancer), and/or other anticancer drugs and therapies. The SMDCs can also be used in a synergistic combination with CAR-expressing cytotoxic lymphocytes as described.

Any SMDC can be prepared by conventional methods of organic synthesis practiced by those skilled in the art. The general reaction sequences outlined below represent general methods useful for preparing the compounds and are not meant to be limiting in scope or utility.

Descriptions of compounds are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art, thereby avoiding inherently unstable compounds.

In some embodiments, the pharmaceutical composition comprises a conjugate, such as at least one SMDC comprising a drug moiety conjugated to a ligand (e.g., a targeting moiety) via a linker, such as PEG or a derivative thereof.

Conjugates can be administered in unit dosage forms and/or compositions containing one or more pharmaceutically acceptable carriers, adjuvants, diluents, excipients, and/or vehicles, and combinations thereof. The term “administering” and its formatives generally refer to any and all means of introducing compounds to the host subject including, but not limited to, by oral, intravenous, intramuscular, subcutaneous, transdermal, inhalation, buccal, ocular, sublingual, vaginal, rectal, and like routes of administration.

Administration of the compounds as salts can be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. Similarly, “pharmaceutically acceptable salt” refers to those salts with counter ions, which may be used in pharmaceuticals. Such salts can include, without limitation: (1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include arginine, benzathine, calcium, choline, diethylamine, diolamine, glycine, lysine, magnesium, meglumine, olamine, potassium, sodium, tromethamine and zinc salts. Hemi-salts of acids and bases also can be formed, for example, hemisulphate and hemicalcium salts.

The conjugates can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, a conjugate, or composition comprising the same, can be administered directly into the blood stream, into muscle, or into an internal organ.

For example, the conjugate can be systemically administered (orally, for example) in combination with a pharmaceutically acceptable vehicle, such as an inert diluent or an assimilable edible carrier. For oral therapeutic administration, the conjugate can be combined with one or more excipients and used in the form of ingestible tablets, buccal tablets, troches, capsules, elixirs, suspensions, syrups, wafers, and the like. The percentage of the compositions and preparations may vary and may be between about 1 to about 99% weight of the active ingredient(s) and a binder, excipients, a disintegrating agent, a lubricant, and/or a sweetening agent (as are known in the art). The amount of the conjugate in such therapeutically useful compositions is such that an effective dosage level will be obtained.

In some embodiments, the SMDC is administered as a composition comprising one or more pharmaceutically acceptable carriers, adjuvants, diluents, excipients, vehicles, or a combination of any of the foregoing.

The preparation of parenteral conjugates/compositions under sterile conditions, for example, by lyophilization, can readily be accomplished using standard pharmaceutical techniques well-known to those skilled in the art. The solubility of a conjugate used in the preparation of a parenteral composition may be increased by the use of appropriate formulation techniques, such as the incorporation of solubility-enhancing agents.

As previously noted, the conjugates/compositions can be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9), but, for some applications, they can be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations may further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents such as parabens, chlorobutanol, phenol, sorbic acid, thimerosal, and the like. In certain cases, it will be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the conjugate in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

For topical administration, it can be desirable to administer a conjugate to the skin as a composition or a formulation in combination with a dermatologically acceptable carrier, which may be a solid or a liquid. In some embodiments, solid carriers include finely divided solids such as talc, clay, microcrystalline cellulose, silica, alumina and the like. Similarly, useful liquid carriers can comprise water, alcohols or glycols or water-alcohol/glycol blends, in which the conjugate can be dissolved or dispersed at effective levels, optionally with the aid of non-toxic surfactants. Additionally or alternatively, adjuvants, such as fragrances and antimicrobial agents, can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

### Chimeric Antigen Receptor (CAR)-Modified Cells

CARs are artificially constructed hybrid receptor proteins or polypeptides containing an antigen binding domain of an antibody (i.e., an antigen recognition domain), such as, e.g., a single chain variable fragment (scFv) linked to signaling or activation domains (i.e., a co-stimulatory domain).

Without being bound to a particular theory or mechanism, it is believed that by eliciting an antigen-specific response against TAA, the CARs provide for one or more of the following: targeting and destroying TAA-expressing cancer cells, reducing or eliminating cancer cells, facilitating infiltration of immune cells to tumor site(s), and enhancing/extending anti-cancer responses. Thus, the disclosure provides a method of destroying malignant cancer cells, which includes contacting one or more cytotoxic lymphocytes (e.g., cytotoxic T cells, NK cells, and LAK cells) engineered to express a CAR with a population of cancer cells that express a TAA disclosed herein, whereby the CAR is produced and binds to a TAA on the cancer cells and the cancer cells are destroyed. As discussed above, treatment of cancer typically involves chemotherapy, radiotherapy, therapeutic monoclonal antibodies, checkpoint inhibitor therapy, among others; however, a high rate of relapse is common in patients that have undergone such treatment across various cancers. The provided compositions include cytotoxic lymphocytes (e.g., cytotoxic T cells, NK cells, and LAK cells) engineered to express a CAR including an antigen recognition domain that contains a monoclonal antibody or an antigen-binding portion thereof directed against a tumor-associated antigen (TAA) disclosed herein. The antigen recognition domain of the CAR can be a whole antibody or an antibody fragment (e.g., scFv). A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen-binding site of an antibody. The VH and VL regions have the same general structure, with each region including four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen recognition and binding.

The terms “fragment of an antibody,” “antibody fragment,” “functional fragment of an antibody,” and “antigen-binding portion” are used interchangeably herein to mean one or more fragments or portions of an antibody that retain the ability to specifically bind to an antigen. The antigen recognition domain of the CAR can contain any TAA-binding antibody fragment capable of targeting the engineered lymphocyte to a target cell. The antibody fragment desirably includes, for example, one or more CDRs, the variable region (or portions thereof), the constant region (or portions thereof), or combinations thereof. Examples of antibody fragments include, but are not limited to: (i) a Fab fragment, which is a monovalent fragment consisting of the VL, VH, CL, and CH1 domains; (ii) a F(ab′)2 fragment, which is a bivalent fragment including two Fab fragments linked by a disulfide bridge at the hinge region; (iii) a Fv fragment consisting of the VL and VH domains of a single arm of an antibody; (iv) scFv, which is a monovalent molecule consisting of the two domains of the Fv fragment (i.e., VL and VH) joined by a synthetic linker which enables the two domains to be synthesized as a single polypeptide chain (see, e.g., Bird et al., Science, 242:423-426 (1988); Huston et al., Proc. Natl. Acad. Sci. USA, 85:5879-5883 (1988); and Osboum et al., Nat. Biotechnol., 16:778 (1998)); and (v) a diabody, which is a dimer of polypeptide chains, wherein each polypeptide chain includes a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. In some embodiments, the antigen recognition domain of the CAR includes a scFv that binds to a TAA disclosed herein.

An antigen-binding portion or fragment of a monoclonal antibody can be of any size so long as the portion binds to the target TAA. In this respect, an antigen binding portion or fragment of the monoclonal antibody directed against a TAA desirably includes one or more CDRs including between about 5 and 18 amino acids (e.g., about 5, 6, 7, 8, 9, 10, 11, 12, 13, 14, 15, 16, 17, 18, or a range defined by any two of the foregoing values).

In some embodiments, the CAR includes an antigen recognition domain that includes a variable region of a monoclonal antibody or an antigen-binding fragment thereof that binds to a TAA. The monoclonal antibody or antigen-binding fragment thereof can be obtained or derived from a mammal, including but not limited to, a mouse, a rat, or a human. In some embodiments, the antigen recognition domain includes a variable region of a mouse or human monoclonal antibody or antigen-binding fragment thereof that binds to a TAA. In this respect, the antigen recognition domain includes a light chain variable region, a heavy chain variable region, or both a light chain variable region and a heavy chain variable region of a mouse or human monoclonal antibody or antigen-binding fragment thereof that binds to a TAA.

In some embodiments, a CAR disclosed herein includes a signal sequence. The signal sequence may be positioned at the amino terminus of the antigen recognition domain (e.g., the variable region of the antibody or antigen-binding fragment thereof). The signal sequence may include any suitable signal sequence. In one embodiment, the signal sequence is a human granulocyte-macrophage colony-stimulating factor (GM-CSF) receptor signal sequence or a CD8a signal sequence. For example, a CAR including a murine scFv can include a GM-CSF signal sequence, while an CAR including a human scFv can include a CD8a signal sequence.

In some embodiments, a CAR disclosed herein includes an extracellular spacer sequence. The extracellular spacer sequence is a short sequence of amino acids that facilitates antibody flexibility (see, e.g., Woof et al., Nat. Rev. Immunol., 4(2): 89-99 (2004)), and may be positioned between the antigen recognition domain (e.g., scFv) and the T cell activation domain. The extracellular spacer sequence can include all or a portion of an extracellular region of any transmembrane protein. In some embodiments, for example, the extracellular spacer sequence is derived from the human CD8a molecule or the human CD28 molecule.

In some embodiments, a CAR disclosed herein also includes a transmembrane domain. The transmembrane domain can be any transmembrane domain derived or obtained from any molecule known in the art. For example, the transmembrane domain can be obtained or derived from a CD8a molecule, CD28 molecule, 2B4 molecule, CD16 molecule, NKp44 molecule, NKp46 molecule, NKG2D molecule, or a DAP12 molecule. CD8 is a transmembrane glycoprotein that serves as a co-receptor for the T cell receptor (TCR) and is expressed primarily on the surface of cytotoxic T cells. The most common form of CD8 exists as a dimer composed of a CD8α and CD8β chain. CD28 is expressed on T cells and provides co-stimulatory signals required for T cell activation. CD28 is the receptor for CD80 and CD86. In some embodiments, the CD8α and CD28 are human CD8α and CD28.

A CAR disclosed herein includes a T cell activation domain. The T cell activation domain includes at least one intracellular (e.g., cytoplasmic) T cell signaling domain (also referred to as a “co-stimulatory domain”). The most common intracellular T cell signaling domain employed in CARs is CD3 zeta (CD3ζ), which associates with TCRs to produce a signal and contains immunoreceptor tyrosine-based activation motifs (ITAMs).

In some embodiments, the T cell activation domain includes multiple (e.g., two or more) intracellular T cell signaling domains. The intercellular T cell signaling domains can be obtained or derived from a CD28 molecule, a CD3ζ molecule or modified versions thereof, the gamma chain of a human high-affinity IgE receptor (FcεRI), a CD27 molecule, an OX40 molecule, a 4-1BB molecule, an ICOS molecule, a CD2 molecule, a CD154 molecule, a DAP10 molecule, an NKG2D molecule, a 2B4 molecule, a DAP10 molecule, a DAP12 molecule, a DNAM1 molecule, a NKG2 molecule, a NKG2D molecule, a CD27 molecule, a CD40L molecule, a CD70 molecule, or other intracellular signaling molecules known in the art. As discussed above, CD28 is a T cell marker important in T cell co-stimulation. 4-1BB, also known as CD137, transmits a potent costimulatory signal to T cells, promoting differentiation and enhancing long-term survival of T lymphocytes. CD27 is a member of the TNF receptor superfamily and is required for generation and long-term maintenance of T cell immunity. The human high-affinity IgE receptor (FcεRI) is a tetrameric receptor complex consisting of one alpha, one beta, and two disulfide bridge connected gamma chains. FcεRI is constitutively expressed on mast cells and basophils and is inducible in eosinophils. In some embodiments, the intracellular T cell signaling domains are human.

In some embodiments the CARs include any one of aforementioned transmembrane domains and any one or more (e.g., 1, 2, 3, or 4) of the aforementioned intracellular T cell signaling domains in any combination. In some embodiments the CAR includes a CD28 transmembrane domain and intracellular T cell signaling domains of CD28 and CD3ζ. In some embodiments, the CAR includes a CD8α transmembrane domain and intracellular T cell signaling domains of CD28, CD3ζ, the gamma chain of FcεRI, and/or 4-1BB. In another embodiment, the CAR can include a CD8α transmembrane domain and intracellular T cell signaling domains of CD28, CD3ζ, and CD27. In some embodiments, the CAR includes a CD28 transmembrane domain and intracellular T cell signaling domains of CD27, 4-1BB, and the gamma chain of FcεRI.

In some embodiments the disclosed CARs and cytotoxic lymphocytes (e.g., cytotoxic T cells, NK cells, and LAK cells) expressing the same are directed to any target molecule of interest (i.e., may incorporate any antigen recognition domain) that includes any one of the aforementioned extracellular spacers, transmembrane domains, and intracellular T cell signaling domains in any combination. In some embodiments, the antigen recognition domain specifically binds to a TAA. In some embodiments, the TAA is selected from the group consisting of CD19, CD20, CD30, CD3, CD4, CD5, BCMA, GD2, LEY, MS4A1, CD22, TNFRSF17, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN.

In some embodiments, the CAR includes: (i) an antigen recognition domain that binds to a TAA; (ii) an extracellular spacer; (iii) a transmembrane domain derived from a human CD8α molecule; and (iv) intracellular T cell signaling domains derived from a human CD3 zeta (CD3ζ) molecule and a human CD28 molecule. In some embodiments, the CAR includes: (i) an antigen recognition domain that binds to a TAA; (ii) an extracellular spacer, (iii) a transmembrane domain derived from a human CD8α molecule, and (iv) intracellular T cell signaling domains derived from a human CD28 molecule, a human CD27 molecule, and a human CD3ζ molecule. In some embodiments, the CAR includes: (i) an antigen recognition domain that binds to a TAA; (ii) an extracellular spacer, (iii) a transmembrane domain derived from a human CD8α molecule, and (iv) intracellular T cell signaling domains derived from a human CD28 molecule, a human CD27 molecule, and the gamma chain of FcεRI. In some embodiments, the CAR includes: (i) an antigen recognition domain that binds to a TAA; (ii) an extracellular spacer, (iii) a transmembrane domain derived from a human CD8α molecule, and (iv) intracellular T cell signaling domains derived from a human CD28 molecule and the gamma chain of FcεRI.

A CAR can include any number of amino acids, provided that the CAR retains its biological activity, e.g., the ability to specifically bind to antigen, mediate cytotoxic activity, detect diseased cells in a mammal, or treat or prevent disease in a mammal, etc. In some embodiments, the CAR includes 50 or more (e.g., 60 or more, 100 or more, or 500 or more) amino acids, but less than 1,000 (e.g., 900 or less, 800 or less, 700 or less, or 600 or less) amino acids. In some embodiments, the CAR is about 50 to about 700 amino acids (e.g., about 70, about 80, about 90, about 150, about 200, about 300, about 400, about 550, or about 650 amino acids), about 100 to about 500 amino acids (e.g., about 125, about 175, about 225, about 250, about 275, about 325, about 350, about 375, about 425, about 450, or about 475 amino acids), or a range defined by any two of the foregoing values.

Provided are functional portions of the CAR. The term “functional portion,” when used in reference to a CAR, refers to any part or fragment of the CAR, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to a nucleic acid sequence encoding the parent CAR, in some embodiments a nucleic acid sequence encoding a functional portion of the CAR encodes a protein including, for example, about 10%, 25%, 30%, 50%, 68%, 80%, 90%, 95%, or more, of the parent CAR.

In some embodiments a functional portion of a CAR contains additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, mediate cytotoxic activity, detect cancer, treat or prevent cancer, etc. More desirably, the additional amino acids enhance the biological activity of the CAR, as compared to the biological activity of the parent CAR.

Also provided are functional variants of the CAR. The term “functional variant” refers to a CAR, a polypeptide, or a protein having substantial or significant sequence identity or similarity to the CAR, which functional variant retains the biological activity of the CAR of which it is a variant. Functional variants encompass, for example, those variants of the CAR (the parent CAR) that retain the ability to recognize target cells to a similar extent, the same extent, or to a higher extent, as the parent CAR. In reference to a nucleic acid sequence encoding the parent CAR, in some embodiments a nucleic acid sequence encoding a functional variant of the CAR is about 10% identical, about 25% identical, about 30% identical, about 50% identical, about 65% identical, about 80% identical, about 90% identical, about 95% identical, or about 99% identical to the nucleic acid sequence encoding the parent CAR.

In some embodiments a functional variant includes the amino acid sequence of the CAR with at least one conservative amino acid substitution. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the same amino acid sub-group, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained.

Alternatively or additionally, in some embodiments the functional variants include the amino acid sequence of the parent CAR with at least one non-conservative amino acid substitution. “Non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution may enhance the biological activity of the functional variant, such that the biological activity of the functional variant is increased as compared to the parent CAR.

In some embodiments the CAR, or a functional portion or variant thereof, includes a synthetic amino acids in place of one or more naturally-occurring amino acids. Such synthetic amino acids are known in the art and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The CAR (including functional portions and functional variants thereof) can be glycosylated, amidated, carboxylated, phosphorylated, esterified, N-acylated, cyclized via, e.g., a disulfide bridge, or converted into an acid addition salt and/or optionally dimerized or polymerized, or conjugated.

### CAR-T Cells

T cells are a principal type of lymphocyte that completes maturation in the thymus and that has various roles in the immune system, including the identification of specific foreign antigens in the body and the activation and deactivation of other immune cells in an MHC class I-restricted manner. T cells can be genetically modified to express CARs. CARs can redirect T cell specificity and reactivity toward a selected target in a non-MHC-restricted manner, exploiting the antigen-binding properties of monoclonal antibodies. The non-MHC-restricted antigen recognition endows T cells expressing CARs with the ability to recognize an antigen independent of antigen processing, thus bypassing a major mechanism of immune escape. Moreover, when expressed in T cells, CARs advantageously do not dimerize with the endogenous T cell receptor (TCR) alpha and beta chains. Thus, provided are modified T cells, which are engineered to express a CAR targeting a TAA.

A T cell suitable for modification with a CAR construct can be any T cell, such as a cultured T cell, e.g., a primary T cell, a T cell from a cultured T cell line, or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell preferably is a human T cell (e.g., isolated from a human). In some embodiments the T cell is of any developmental stage, including but not limited to, a CD4⁺/CD8⁺ double positive T cell, a CD4⁺ helper T cell, e.g., Th₁ and Th₂ cells, a CD8⁺ T cell (e.g., a cytotoxic T cell), a tumor infiltrating cell, a memory T cell, a naïve T cell, and the like. In some embodiments, the T cell is a CD8⁺ T cell or a CD4⁺ T cell. T cell lines are available from, e.g., the American Type Culture Collection (ATCC, Manassas, VA) and the German Collection of Microorganisms and Cell Cultures (DSMZ) and include, for example, Jurkat cells (ATCC TIB-152), Sup-T1 cells (ATCC CRL-1942), RPMI 8402 cells (DSMZ ACC-290), Karpas 45 cells (DSMZ ACC-545), and derivatives thereof. Exemplary TAA targets for CAR-T cells include, without limitation, CD19, CD20, CD30, BCMA, GD2, LEY, MS4A1, CD22, TNFRSF17, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN.

### CAR-NK Cells

Natural killer (NK) cells represent an important part of innate immunity. Unlike T cells, NK cells can initiate anti-tumor cytotoxicity without prior sensitization and may potentially have fewer complications due to cytokine release syndrome, and on-target/off-tumor effects. NK cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of interleukin 2 (IL-2), strong cytotoxic reactivity develops. NK cells can be detected by specific surface markers, such as CD16, CD56, and CD8 in humans. NK cells do not express T-cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors. When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-γ, tumor necrosis factor-α and granulocyte-macrophage colony-stimulating factor (GM-CSF), which activate both innate and adaptive immune cells as well as other cytokines. Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses. Because of shared signaling activation mechanisms in T-cells and NK-cells, the CAR construct containing CD3-ζ activation domain can also activate NK cells.

Accordingly, provided are NK cells engineered to express a CAR construct (CAR-NK cell). CAR-NK cells can be targeted to any suitable TAA. In certain embodiments, CAR-NK cells are targeted to a TAA selected from the group consisting of CD3, CD4, and CD5. In certain embodiments, CAR-NK cells can include a co-stimulatory domain described in the foregoing sections. In certain embodiments, CAR-NK cells include a co-stimulatory domain selected from the group consisting of a co-stimulatory domain derived from a CD28 molecule, a 2B4 molecule, a 4-1BB molecule, a DNAM1 molecule, a DAP10 molecule, a DAP12 molecule, a NKG2 molecule, and a NKG2D molecule. In certain embodiments, CAR-NK cells can include a transmembrane domain described in the foregoing sections. In some embodiments, CAR-NK cells include a transmembrane domain selected from the group consisting of a transmembrane domain derived from a CD8 molecule, a CD28 molecule, a DAP12 molecule, a 2B4 molecule, a NKp44 molecule, a NKp46 molecule, and a NKG2D molecule.

A NK cell may be any NK cell, such as an NK cell derived from cord blood, peripheral blood, bone marrow, CD34⁺ cells, or iPSCs. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well-known in the art. Specifically, the NK cells may be isolated from cord blood (CB), peripheral blood (PB), bone marrow, or stem cells. In some embodiments, the immune cells are isolated from pooled CB. In some embodiments CB is pooled from 2, 3, 4, 5, 6, 7, 8, 10, or more units. In some embodiments the immune cells are autologous or allogeneic. In some embodiments the isolated NK cells are haplotype matched for the subject to be administered the cell therapy. NK cells can be detected by specific surface markers, such as CD16, CD56, and CD8 in humans with absence of CD3 expression. In some embodiments, the starting population of NK cells is obtained by isolating mononuclear cells using Ficoll density gradient centrifugation. The cell culture may be depleted of any cells expressing CD3, CD14, and/or CD19 cells and may be characterized to determine the percentage of CD56⁺/CD3⁻ cells or NK cells.

### CAR-LAK Cells

A lymphokine-activated killer (LAK) cell can be any LAK cell, such as a LAK cell derived from PBMCs. LAK cells represent a composite population of CD3⁻ NK cells and CD3⁺ T cells, of both the CD4 and CD8 subsets. Characteristic of LAK cells is their capacity to lyse a variety of tumor cells in a non-MHC-restricted fashion. In addition, LAK cells can kill class I negative target cells, such as Daudi and K562, which serve as general standards for identification of non-MHC-restricted cytotoxic effector cells. Separation of LAK populations into CD3- and CD3⁺ fractions revealed that both NK cells and T cells could lyse class I negative target cells and a variety of different tumor cells. Furthermore, the LAK-T cells can also be inhibited by expression of particular MHC class I molecules on target cells. In some embodiments, the LAK cell is a LAK-T cell. In some embodiments, the LAK-T cell is an activated LAK-T cell of the CD4+ or CD8+ subtype.

### Nucleic Acids

The disclosure further provides an isolated or purified nucleic acid sequence encoding the CAR. “Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides.

A CAR disclosed herein can be generated using methods known in the art. For example, nucleic acid sequences, polypeptides, and proteins can be recombinantly produced using standard recombinant DNA methodology (see, e.g., Sambrook et al., Molecular Cloning: A Laboratory Manual, 3^{rd} ed., Cold Spring Harbor Press, Cold Spring Harbor, N.Y., 2001; and Ausubel et al., Current Protocols in Molecular Biology, Greene Publishing Associates and John Wiley & Sons, N Y, 1994). Further, a synthetically produced nucleic acid sequence encoding the CAR can be isolated and/or purified from a source, such as a plant, a bacterium, an insect, or a mammal, e.g., a rat, a human, etc. Methods of isolation and purification are well-known in the art. Alternatively, the nucleic acid sequences described herein can be commercially synthesized. In this respect, the nucleic acid sequence can be synthetic, recombinant, isolated, and/or purified.

### Nucleic Acid Vectors

Also provided is a vector including the nucleic acid sequence encoding the CAR. In some embodiments the vector is a plasmid, a cosmid, a viral vector (e.g., retroviral or adenoviral), or a phage. Suitable vectors and methods of vector preparation are well-known in the art.

In addition to the nucleic acid sequence encoding a CAR, the vector includes, in some embodiments, regulatory sequences, such as, e.g., promoters, enhancers, polyadenylation signals, transcription terminators, internal ribosome entry sites (IRES), and the like, that provide for the expression of the nucleic acid sequence in a host cell. Exemplary regulatory sequences suitable for incorporation into expression vectors are well-known in the art.

A large number of promoters, including constitutive, inducible, and repressible promoters, from a variety of different sources are well-known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system, the Ecdysone inducible system, the T-REX™ system (Invitrogen, Carlsbad, CA), LACSWITCH™ System (Stratagene, San Diego, CA), and the Cre-ERT tamoxifen-inducible recombinase system.

The term “enhancer” refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well-known in the art and are available as or within cloned polynucleotides (from, e.g., depositories such as the ATCC as well as other commercial or individual sources). A number of polynucleotides including promoters (such as the commonly used CMV promoter) also include enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers.

In some embodiments the vector includes a “selectable marker gene.” The term “selectable marker gene” refers to a nucleic acid sequence that allows cells expressing the nucleic acid sequence to be specifically selected for or against in the presence of a corresponding selective agent. Suitable selectable marker genes are known in the art.

In some embodiments, the vector is an “episomal expression vector” or “episome,” which is able to replicate in a host cell and persists as an extrachromosomal segment of DNA within the host cell in the presence of appropriate selective pressure. Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pBK-CMV from Stratagene (La Jolla, CA) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site-specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Invitrogen (Carlsbad, CA), and pCI or pFN10A (ACT) FLEXI™ from Promega (Madison, WI).

Viral vectors can also be used in conjunction with the methods and compositions disclosed herein. Representative viral expression vectors include, but are not limited to, the adenovirus-based vectors (e.g., the adenovirus-based Per.C6 system available from Crucell, Inc. (Leiden, Netherlands)), lentivirus-based vectors (e.g., the lentiviral-based pLP1 from Life Technologies (Carlsbad, CA)), and retroviral vectors (e.g., the pFB-ERV plus pCFB-EGSH from Stratagene (La Jolla, CA)). In some embodiments, the viral vector is a lentivirus vector.

The vector including a nucleic acid encoding a CAR can be introduced into a host cell that can express the CAR, including any suitable prokaryotic or eukaryotic cell. Preferred host cells are those that can be easily and reliably grown, have reasonably fast growth rates, have well-characterized expression systems, and can be transformed or transfected easily and efficiently.

### Host Cells

The term “host cell” refers to any type of cell that can contain the expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell can be a mammalian cell. The host cell is a human cell in some embodiments. The host cell can be of any cell type, can originate from any type of tissue, and can be of any developmental stage. In some embodiments, the host cell is a peripheral blood lymphocyte (PBL), a peripheral blood mononuclear cell (PBMC), a NK cell, a LAK cell, or a T cell. In some embodiments, the host cell is a T cell. In some embodiments, the host cell is a NK cell. In some embodiments, the host cell is a LAK cell. Methods for selecting suitable mammalian host cells and methods for transformation, culture, amplification, screening, and purification of cells are known in the art.

An isolated cytotoxic lymphocyte (e.g., cytotoxic T cell, NK cell, and LAK cell), which expresses a nucleic acid sequence encoding a CAR, is provided.

Methods for Introducing CAR-Encoding Nucleic Acids into Host Cells

A nucleic acid sequence encoding the CAR may be introduced into a cell by “transfection,” “transformation,” or “transduction.” The terms “transfection,” “transformation,” and “transduction” refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

One or more isolated cytotoxic lymphocytes (e.g., T cells, NK cells, or LAK cells) expressing a nucleic acid sequence encoding a CAR can be contacted with a population of cancer cells that express a TAA ex vivo, in vivo, or in vitro. The method, in some embodiments, involves ex vivo and in vivo components. In this regard, for example, the isolated and CAR-engineered cytotoxic lymphocytes cells described above can be cultured ex vivo under conditions to express a nucleic acid sequence encoding the CAR, and then directly transferred into a mammal (e.g., a human) affected by a cancer. Such a cell transfer method is referred to in the art as “adoptive cell transfer (ACT),” in which immune-derived cells are passively transferred into anew recipient host to transfer the functionality of the donor immune-derived cells to the new host. Adoptive cell transfer methods to treat various types of cancers are known in the art and disclosed in, for example, Gattinoni et al., Nat. Rev. Immunol., 6(5): 383-393 (2006); June, C H, J. Clin. Invest., 117(6): 1466-76 (2007); Rapoport et al., Blood, 117(3): 788-797 (2011); and Barber et al., Gene Therapy, 18: 509-516 (2011)).

### Administration of CAR-Engineered Cells

When the CAR-engineered cells are administered to a mammal, the cells can be allogeneic or autologous to the mammal. In “autologous” administration methods, cells (e.g., blood-forming stem cells or lymphocytes) are removed from a mammal, stored (and optionally modified), and administered back to the same mammal. In “allogeneic” administration methods, a mammal receives cells (e.g., blood-forming stem cells or lymphocytes) from a genetically similar, but not identical, donor. Preferably, the cells are autologous to the mammal.

The CAR-engineered cells desirably are administered to a human in the form of a composition, such as a pharmaceutical composition comprising a population of the engineered cells expressing a CAR and one or more (e.g., 1, 2, 3, or more) of the SMDC agents. Alternatively, a nucleic acid sequence encoding the CAR, or a vector including the CAR-encoding nucleic acid sequence, can be formulated into a composition, such as a pharmaceutical composition, and administered to a human. In addition to the one or more (e.g., 1, 2, 3, or more) of the SMDC agents, a nucleic acid sequence encoding the CAR, and/or a population of cytotoxic lymphocytes engineered to express the CAR, the pharmaceutical composition can further include other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In certain embodiments, the pharmaceutical composition includes an isolated T cell, which expresses the CAR, more preferably a population of T cells which expresses the CAR. In certain embodiments, the pharmaceutical composition includes an isolated NK cell, which expresses the CAR, more preferably a population of NK cells which expresses the CAR. In certain embodiments, the pharmaceutical composition includes an isolated LAK cell, which expresses the CAR, more preferably a population of LAK cells which expresses the CAR.

The choice of carrier will be determined in part by the particular CAR, CAR-encoding nucleic acid sequence, vector, or host cells expressing the CAR, as well as by the particular method used to administer the CAR-encoding nucleic acid sequence, vector, or host cells expressing the CAR. Accordingly, there are a variety of suitable formulations of the pharmaceutical composition. For example, the pharmaceutical composition can contain preservatives. Suitable preservatives may include, for example, methylparaben, propylparaben, sodium benzoate, and benzalkonium chloride. A mixture of two or more preservatives optionally can be used. The preservative or mixtures thereof are typically present in an amount of about 0.0001% to about 2% by weight of the total composition.

A typical amount of host cells administered to a mammal (e.g., a human) can be, for example, in the range of one million to 100 billion cells; however, amounts below or above this exemplary range are within the scope of the disclosure. For example, the daily dose of host cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values).

### Modified T Cell Receptor (TCR)-T Cells

T cell receptors (TCRs) are naturally expressed by CD4⁺ and CD8⁺ T cells. TCRs are designed to recognize short peptide antigens that are displayed on the surface of antigen presenting cells in complex with Major Histocompatibility Complex (MHC) molecules (in humans, MHC molecules are also known as human leukocyte antigens (HLA)). CD8⁺ T cells, which are also termed cytotoxic T cells, specifically recognize peptides bound to MHC class I and are generally responsible for finding and mediating the destruction of diseased cells. CD8⁺ T cells can destroy cancerous as well as virally infected cells; however, the affinity of TCRs expressed by cancer-specific T cells in the natural repertoire are typically low because of thymic selection, meaning that cancerous cells frequently escape detection and destruction.

The native TCR is a heterodimeric cell surface protein of the immunoglobulin superfamily which is associated with invariant proteins of the CD3 complex involved in mediating signal transduction. TCRs exist in αβ and γδ forms, which are structurally similar but have distinct anatomical locations and functions. MHC class I and class II ligands are also immunoglobulin superfamily proteins that are specialized for antigen presentation, with a highly polymorphic peptide binding site which enables them to present a diverse array of short peptide fragments at the antigen-presenting cell (APC) surface. The extracellular portion of native heterodimeric αβ and γδ TCRs consists of two polypeptides, each of which has a membrane-proximal extracellular constant domain and a membrane-distal variable region. Each of the extracellular constant domain and variable region includes an intra-chain disulfide bond. The variable regions contain the highly polymorphic loops analogous to the complementarity determining regions (CDRs) of antibodies. CDR3 of αβ TCRs interact with the peptide presented by MHC, and CDRs 1 and 2 of αβ TCRs interact with the peptide and the MHC.

Immunotherapeutic approaches aimed at promoting cancer recognition by T cells offer a highly promising strategy for the development of effective anti-cancer treatments. A recent proposed variation of T cell adoptive therapy is the use of gene therapy techniques to introduce TCRs specific for known cancer-specific MHC-peptide complexes into the T cells of cancer patients. Thus, compositions and methods for immunotherapy using engineered TCR-T cells in combination with one or more (e.g., 1, 2, 3, or more) SMDC agents are provided. Compositions comprising engineered TCR-T cells may be used to target abnormal cells presenting cancer-specific MHC complexes.

The provided compositions and methods include an SMDC and a population of engineered TCR-expressing cytotoxic lymphocytes (e.g., T cells, such as, e.g., cytotoxic T cells) that target and bind to a TAA disclosed herein.

TCRs may be naturally occurring, non-naturally occurring, and/or engineered. TCRs may have more than one mutation present in the α chain variable domain and/or the β chain variable domain relative to the parental TCR. “Engineered TCR” and “mutant TCR” are used synonymously herein and generally mean a TCR, which has one or more mutations introduced relative to the parental TCR, in particular, in the α chain variable domain and/or the β chain variable domain thereof. In some embodiments, the α chain variable domain and/or the β chain variable domain incorporates one or more (e.g., 1, 2, 3, 4, 5, 6, 7, 8, 9, 10, or more) mutations.

The TCRs may be ap heterodimers. TCRs may be in single chain format. Single chain formats include, but are not limited to, ap TCR polypeptides of the Vα-L-Vμ, Vβ-L-Vα, Vα-Cα-L-Vβ, Vα-L-Vβ-Cμ, or Vα-Vα-L-Vβ-Cμ types, wherein Vα and Vβ are TCR α and β variable regions respectively, Cα and Cβ are TCR α and β constant regions respectively, and L is a linker sequence. One or both of the constant domains may be full length, or they may be truncated as described above, and/or contain mutations. The α chain extracellular constant may have an asparagine (N) or a lysine (K) residue at position 4 due to a natural polymorphism. In some embodiments, single chain TCRs have an introduced disulfide bond between residues of the respective constant domains. As will be apparent to those skilled in the art, it may be possible to truncate the sequences provided at the C-terminus and/or N-terminus thereof, by, e.g., 1, 2, 3, 4, 5 or more residues, without substantially affecting the binding characteristics of the TCR.

α-β heterodimeric TCRs usually comprise an a chain TRAC constant domain sequence and/or a β chain TRBC1 or TRBC2 constant domain sequence. The α and β chain constant domain sequences may be modified by truncation or substitution to delete the native disulfide bond between Cys4 of exon 2 of TRAC and Cys2 of exon 2 of TRBC1 or TRBC2. The α and/or β chain constant domain sequence(s) may be modified by, e.g., substitution of cysteine residues for Thr 48 of TRAC and Ser 57 of TRBC1 or TRBC2, the said cysteines forming a disulfide bond between the α and β constant domains of the TCR. TRBC1 or TRBC2 may additionally include a cysteine to alanine mutation at position 75 of the constant domain and an asparagine to aspartic acid mutation at position 89 of the constant domain. The constant domain may additionally or alternatively contain further mutations, substitutions or deletions relative to the native TRAC and/or TRBC1/2 sequences. The term TRAC and TRBC1/2 encompasses natural polymorphic variants, such as, e.g., a N to K substitution at position 4 of TRAC. Also provided are variants, fragments and derivatives of the TCRs.

In some embodiments, a TCR targets a TAA expressed in a cancer cell. In some embodiments, the TAA is a cell surface protein. In some embodiments, the TAA is a transmembrane protein. In some embodiments, the TAA is an intracellular protein. In some embodiments, the TAA is identified by isolating cancer cells from a subject (e.g., a human) and identifying a TAA selectively expressed or upregulated in the cancer cells. After the target is confirmed, a TCR display library can be constructed to screen TCRs with high affinity and specificity for the TAA. Generally, any antigen capable of being presented by MHC molecules can be recognized by TCR-T cells, including intracellular and cell surface antigens. In some embodiments, the TAA is selected from the group consisting of CD19, CD20, CD30, CD3, CD4, CD5, BCMA, GD2, LEY, MS4A1, CD22, TNFRSF17, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN.

For some purposes, the TCRs may be aggregated into a complex comprising several TCRs to form a multivalent TCR complex. There are a number of human proteins that contain a multimerization domain that may be used in the production of multivalent TCR complexes. For example, the tetramerization domain of p53 has been utilized to produce tetramers of scFv antibody fragments, which exhibit increased serum persistence and significantly reduced off-rate toxicity compared to the monomeric scFv fragment. Hemoglobin also has a tetramerization domain that could potentially be used for this kind of application.

As is well-known in the art, TCRs may be subject to post-translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (e.g., oligosaccharide type, covalent linkage and total number of attachments) can influence protein function. Therefore, when producing recombinant proteins, controlling glycosylation is often desirable. Controlled glycosylation has been used to improve antibody-based therapeutics. Such modifications are desirable, since glycosylation can improve pharmacokinetics, reduce immunogenicity and more closely mimic a native human protein.

For administration to patients, the TCRs, nucleic acids and/or cells (usually associated with a detectable label or therapeutic agent), may be provided in a pharmaceutical composition, together with one or more SMDC agents and a pharmaceutically acceptable carrier or excipient. Therapeutic or imaging TCRs will usually be supplied as part of a sterile, pharmaceutical composition, which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

Since the TCRs have utility in adoptive T cell therapy, a non-naturally occurring and/or purified and/or or engineered cell, especially a T-cell, presenting a TCR is provided. Also provided is an expanded population of T cells presenting a TCR. There are a number of methods suitable for the transfection of T cells with nucleic acid (such as, e.g., DNA, cDNA or RNA) encoding the TCRs. T cells expressing the TCRs will be suitable for use in adoptive therapy-based treatment of cancer. As will be known to those skilled in the art, there are a number of suitable methods by which adoptive therapy can be carried out.

In some embodiments, each CAR is a fusion protein comprising a recognition region, a co-stimulation domain, and an activation signaling domain, and the CAR binds a cell-surface antigen on an immunosuppressive cell or a cancer cell with specificity.

In some embodiments, the recognition region is a scFv region of an anti-FITC (fluorescein isothiocyanate) antibody, the co-stimulation domain is CD28, and the activation signaling domain is a T cell CD3ζ chain. In some embodiments the recognition region is a scFv region of an anti-CD19 antibody, the co-stimulation domain is CD137 (4-1BB), and the activation signaling domain is a T cell CD3ζ chain.

In some embodiments, the recognition region is a scFv region of an anti-CD19 antibody, the co-stimulation domain is CD28, and the activation signaling domain is a T cell CD3ζ chain.

In some embodiments, the recognition region is a scFv of an antibody that binds to a cell-surface antigen with high specificity.

In some embodiments each CAR comprises a recognition region, a co-stimulation domain, and an activation signaling domain.

In some embodiments the recognition region of the CAR is a scFv of an antibody that binds to a cell-surface antigen with high specificity.

In some embodiments the CAR selectively binds a cell-surface antigen on an immunosuppressive cell or a cancer cell.

In some embodiments the CAR selectively binds a cell-surface antigen on an immunosuppressive cell or a cancer cell with specificity.

In some embodiments the cell-surface antigen is a tumor-associated antigen (TAA).

In some embodiments the TAA is selected from the group consisting of CD19, CD20, CD30, CD3, CD4, CD5, BCMA, GD2, LEY, MS4A1, CD22, TNFRSF17, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN.

In some embodiments the TAA is CD19.

In some embodiments the co-stimulation domain of the CAR is CD27. CD40L, CD70, 2B4, DNAM1, DAP12, DAP10, NKG2, NKG2D, CD28, CD137 (4-1BB), CD134 (OX40), or CD278 (ICOS).

In some embodiments the activation signaling domain of the CAR is a T cell CD34 chain or a Fc receptor γ.

In some embodiments the cytotoxic lymphocyte is autologous to the individual.

In some embodiments the cytotoxic lymphocyte is allogeneic.

In some embodiments the cytotoxic lymphocyte is heterologous to the individual.

### Methods of Use & Combinations

Methods and combinations for treating a cancer (e.g., a solid tumor) are provided. The term “combination” generally refers to any product comprising more than one ingredient, including one or more of the compounds (e.g., a SMDC or a pharmaceutically acceptable salt of the foregoing). It is to be understood that the compositions can be prepared from isolated compounds or from salts, solutions, hydrates, solvates, and other forms of the compounds. It is appreciated that certain functional groups, such as the hydroxy, amino, and like groups, can form complexes with water and/or various solvents, in the various physical forms of the compounds. It will also be understood that, in certain circumstances, the compounds (and compositions comprising the compounds) can be prepared from various amorphous, non-amorphous, partially crystalline, crystalline, and/or other morphological forms of the compounds, and the compositions can be prepared from various hydrates and/or solvates of the compounds. Accordingly, pharmaceutical compositions that recite the compounds include each of, or any combination of, or individual forms of, the various morphological forms and/or solvate or hydrate forms of the compounds.

In certain embodiments, the combination can be a synergistic combination that, when administered to a subject (e.g., using a therapeutically effective amount) provides an increased cytotoxic effect on a cancer in the subject as compared to administration to the subject of a single compound of the combination alone. In certain embodiments, administration of the combination (e.g., a therapeutically effective amount of the compound(s) (e.g., at least one SMDC or a pharmaceutically acceptable salt thereof) and a CAR-expressing cytotoxic lymphocyte) inhibits cancer-associated fibroblast activity in the subject and increases the efficacy of the CAR-expressing lymphocyte against the cancer as compared to CAR-expressing cytotoxic lymphocytes administered alone.

A combination for treating cancer comprises one or more compounds comprising the SMDCs (or pharmaceutically acceptable salts thereof) and/or one or more compositions, and a composition comprising cytotoxic lymphocytes expressing a CAR or a vector comprising a promoter operatively linked to a nucleic acid sequence encoding the CAR.

Compounds and compositions can be administered in unit dosage forms and/or compositions containing one or more pharmaceutically acceptable carriers, adjuvants, diluents, excipients, and/or vehicles, and combinations thereof. The term “administering,” and its formatives, generally refer to any and all means of introducing the compounds and compositions (e.g., the CAR-expressly cytotoxic lymphocyte compositions and/or SMDC compounds or compositions) to a cell, tissue, organ, or biological fluid of a subject.

In some embodiments, the SMDC is administered as a composition comprising one or more pharmaceutically acceptable carriers, adjuvants, diluents, excipients, vehicles, or a combination of any of the foregoing.

Administration of the compounds and compositions as salts may be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable.

The compounds and compositions can be formulated as pharmaceutical compositions and/or administered to a subject, such as a human patient, in a variety of forms adapted to the chosen route of administration. Indeed, the adaptor compound, or the pharmaceutically acceptable salt thereof, or the activity modifying compound, or the pharmaceutically acceptable salt thereof, or the CAR-expressing cytotoxic lymphocyte composition, or the vector composition (including, for example, the lentiviral particles hereof) can be administered to a subject using any suitable method known in the art. In one aspect, the SMDC compound, or the pharmaceutically acceptable salt thereof can be administered in unit dosage forms and/or formulations containing conventional nontoxic pharmaceutically acceptable carriers, adjuvants, and vehicles.

Further, the SMDC compound(s), or the pharmaceutically acceptable salt(s) thereof, or the CAR-expressing cytotoxic lymphocyte composition, or a vector composition can be administered orally or directly into the blood stream, into muscle, or into an internal organ. In some embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In some embodiments, means for parenteral administration include needle (including microneedle) injectors, needle-free injectors and infusion techniques. It will be appreciated that the compounds and compositions hereof can be formulated for the desired administration modality as well.

In some embodiments, the SMDC and the CAR-expressing cytotoxic lymphocytes are administered to the subject via a mode of administration selected from the group consisting of intravenously, intramuscularly, intraperitoneally, and subcutaneously, wherein a mode of administration of the SMDC is independent of a mode of administration of the CAR-expressing cytotoxic lymphocytes.

In some embodiments, the SMDC and the CAR-expressing cytotoxic lymphocytes are administered to the subject via intravenous injection.

For example, parenteral formulations are typically aqueous solutions and can contain carriers or excipients such as salts, carbohydrates and buffering agents (preferably at a pH of from 3 to 9), but can also be formulated, where suitable, as a sterile non-aqueous solution or as a dried form to be used in conjunction with a suitable vehicle such as sterile, pyrogen-free water or sterile saline. In some embodiments, any of the liquid formulations are adapted for parenteral administration. The preparation under sterile conditions, by lyophilization to produce a sterile lyophilized powder for a parenteral formulation, can readily be accomplished using standard pharmaceutical techniques well-known to those skilled in the art.

The pharmaceutical dosage forms of the SMDC compound(s) that are suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions, or by the use of surfactants. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the active component in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

Useful dosages of the compounds can be determined by comparing their in vitro activity and the in vivo activity in animal models. Methods of the extrapolation of effective dosages in mice and other animals to human subjects are known in the art. Indeed, the dosage of the SMDC compound can vary significantly depending on the condition of the subject, the cancer type being treated, how advanced the pathology is, the route of administration of the compound and tissue distribution, and the possibility of co-usage of other therapeutic treatments (such as radiation therapy or additional drugs in combination therapies). The amount of the compositions and/or compound(s) required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. “Therapeutically effective amount,” “therapeutically effective dose,” “therapeutically effective,” or “prophylactically effective amount” is defined as (unless specifically stated otherwise) an amount of a conjugate (e.g., the SMDC) or pharmaceutical composition that, when administered either one time or over the course of a treatment cycle affects the health, well-being or mortality of a subject (e.g., and without limitation, delays the onset of and/or reduces the severity of one or more of the symptoms associated with the cancer).

Also provided is a method of treating a subject for cancer. The method comprises administering, by any method, any of the compounds, compositions, and/or combinations to the patient, whereupon the patient is treated for cancer. The compounds, compositions and/or combinations can be administered, for example, in a therapeutically effective amount. In certain embodiments, administration of the SMDC (or pharmaceutically acceptable salt thereof) and the CAR-T cell therapy (e.g., CAR-expressing cytotoxic lymphocytes) and/or a combination hereof inhibits cancer-associated fibroblast activity in the patient and increases the efficacy of the administered CAR-T cell therapy and/or CAR-expressing cytotoxic lymphocytes against the cancer as compared to the efficacy of the CAR-T cell therapy and/or CAR-expressing cytotoxic lymphocytes administered in the absence of the administration of a therapeutically effective amount of the SMDC (or a pharmaceutically acceptable salt thereof).

In some embodiments, the cancer is additionally imaged prior to administration to the subject of the SMDC compound(s), or the pharmaceutically acceptable salts thereof, or the CAR-expressing cytotoxic lymphocyte composition. The cancer additionally, or alternatively, in some embodiments is imaged during or after administration to assess metastasis, for example, and the efficacy of treatment. In some embodiments imaging can occur by positron emission tomography (PET) imaging, magnetic resonance imaging (MRI), or single-photon-emission computed tomography (SPECT)/computed tomography (CT) imaging. The imaging method can be any suitable imaging method known in the art.

In some embodiments, the method further comprises imaging the solid tumor cancer prior to or during administering of the SMDC and CAR-expressing cytotoxic lymphocyte.

In some embodiments, the cancer being treated is a tumor. In some embodiments, the cancer is malignant. In some embodiments, the cancer is a solid tumor cancer.

In some embodiments the cancer is a cancer of the brain, thyroid, lung, pancreas, kidney, stomach, gastrointestinal stroma, endometrium, breast, cervix, ovary, colon, prostate, or head and neck. In some embodiments, the cancer is a leukemia, a lymphoma, or other blood-related cancer.

In some embodiments the cancer is a folate receptor-expressing cancer. In some embodiments the ligand (A) is a radical of raltitrexed or a radical of 5-methyltetrahydrofolate, and the cancer is a folate receptor-expressing cancer. In some embodiments the cancer is a folate receptor α-expressing cancer, a folate receptor β-expressing cancer, a folate receptor δ-expressing cancer, or a cancer mediated by folate receptor α, β, or δ-expressing immune cells. In some embodiments the ligand (A) is a radical of raltitrexed or a radical of 5-methyltetrahy drofolate, and the cancer is a folate receptor α-expressing cancer, a folate receptor β-expressing cancer, a folate receptor δ-expressing cancer, or a cancer mediated by folate receptor α, ρ, or δ-expressing immune cells.

In some embodiments the cancer is a cancer expressing fibroblast activation protein (FAP), or a cancer with cancer associated fibroblasts which express FAP. In some embodiments the ligand (A) is a group comprising formula (X), and the cancer is a cancer expressing FAP or a cancer with cancer associated fibroblasts which express FAP.

In some embodiments, the method further comprises administering a first therapeutically effective amount of the SMDC and a second therapeutically effective amount of the CAR-expressing cytotoxic lymphocytes.

In some embodiments, administering the SMDC comprises administering a dose of the SMDC to the subject at least three times per week.

In some embodiments, administering the SMDC comprises administering a dose of the SMDC to the subject five times per week.

In some embodiments, the first therapeutically effective amount of the SMDC and the second therapeutically effective amount of the CAR-expressing cytotoxic lymphocytes are administered simultaneously or sequentially, in either order.

In some embodiments, the method increases an amount of myeloid cells exhibiting an immune-stimulating phenotype in a tumor microenvironment (TME) of the subject as compared to an amount of myeloid cells exhibiting an immunosuppressive phenotype in the TME.

In the compounds, compositions, combinations, and methods, all embodiments of the SMDC (including, without limitation, the drug moiety or pharmaceutically acceptable salt thereof, and/or the ligand/targeting moiety thereof), the CAR-expressing cytotoxic lymphocyte compositions, and the vector compositions are applicable, including, but not limited to, the linker embodiments.

Uses of the SMDCs or pharmaceutically acceptable salts hereof in the manufacture of a medicament for the treatment of cancer are also provided, where the treatment comprises administering to a patient the SMDC(s) in combination with CAR T-cell therapy. The SMDC(s) can comprise any SMDC described herein, and the CAR T-cell therapy can comprise administration of any CAR-expressing cytotoxic lymphocyte described herein.

### General

All patents, patent application publications, journal articles, textbooks, and other publications mentioned in the specification are indicative of the level of skill of those in the art to which the disclosure pertains.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

When ranges are used herein for physical properties, such as molecular weight, or chemical properties, such as chemical formulae, all combinations and sub-combinations of ranges and specific embodiments therein are intended to be included.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

The term “receptor” refers to a chemical structure in biological systems that receives and transmits signals.

Unless otherwise expressly stated, depicted structures include all stereochemical forms of the structure, i.e., the right-hand (R) and left-hand (S) configurations of each asymmetric center. Therefore, single stereochemical isomers, as well as enantiomeric and diastereomeric mixtures, are within the scope of the present disclosure.

One of ordinary skill in the art will further appreciate that the above SMDCs can be “deuterated,” meaning one or more hydrogen atoms can be replaced with deuterium. As deuterium and hydrogen have nearly the same physical properties, deuterium substitution is the smallest structural change that can be made. Replacement of hydrogen with deuterium can increase stability in the presence of other drugs, thereby reducing unwanted drug-drug interactions, and can significantly lower the rate of metabolism (due to the kinetic isotope effect). By lowering the rate of metabolism, half-life can be increased, toxic metabolite formation can be reduced, and the dosage amount and/or frequency can be decreased.

The term “alkylene,” by itself or as part of another substituent means, unless otherwise stated, a divalent radical derived from an alkyl, as exemplified, but not limited to, —CH₂CH₂CH₂CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combination(s) thereof, consisting of at least one carbon atom and at least one heteroatom selected from the group consisting of oxygen, nitrogen, phosphorous, silicone, and sulfur, and wherein the nitrogen and sulfur atoms may optionally be oxidized, and the nitrogen heteroatom may optionally be quarternized. The heteroatom(s) oxygen, nitrogen, phosphorous, sulfur, and silicone can be placed at any interior position of the heteroalkyl group or at the position at which the alkyl group is attached to the remainder of the molecule. Examples include, without limitation, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂=CH—O—CH₃, —Si(CH₃)₃, —CH₂—CH═N—OCH₃, —CH═CH— N(CH₃)—CH₃, —O—CH₃, —O—CH₂—CH₃, and —CN. Up to two heteroatoms can be consecutive, such as, for example, —CH₂—NH—OCH₃.

Similarly, the term “heteroalkylene,” by itself or as part of another substituent, means (unless otherwise stated) a divalent radical derived from heteroalkyl, as exemplified, but not limited by, —CH₂—CH₂—S—CH₂—CH₂ and —CH₂—S—CH₂—CH₂—NH—CH₂. For heteroalkylene groups, heteroatoms can also occupy either terminus or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “polypeptide,” “peptide,” and “protein” are used interchangeably herein to refer to a polymer of amino acid residues, a polypeptide, or a fragment of a polypeptide, peptide, or fusion polypeptide. The terms apply to amino acid polymers in which one or more amino acid residues is/are an artificial chemical mimetic of a corresponding naturally occurring amino acid, as well as to naturally occurring amino acid polymers and non-naturally occurring amino acid polymers.

Pharmaceutical compositions are also provided. As used herein, the term “composition” generally refers to any product comprising more than one ingredient, e.g., one or more conjugates such as SMDCs. It is to be understood that the compositions can be prepared from isolated conjugates or from salts, solutions, hydrates, solvates, and other forms. It is appreciated that certain functional groups, such as the hydroxy, amino, and like groups, can form complexes with water and/or various solvents in the various physical forms of the conjugates. It is also to be understood that the compositions can be prepared from various amorphous, non-amorphous, partially crystalline, crystalline, and/or other morphological forms of the conjugates, and the compositions can be prepared from various hydrates and/or solvates of the conjugates. Accordingly, pharmaceutical compositions that recite the conjugates include each of, or any combination of, or individual forms of, the various morphological forms and/or solvate or hydrate forms of the conjugates.

As used herein, a “subject” is a mammal, preferably a human, but it can also be a non-human animal (including, without limitation, a laboratory, an agricultural, a domestic, or a wild animal). Thus, the methods, compounds, and compositions are applicable to both human and veterinary disease and applications. In various aspects, the subject can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, or a goat, or a wild animal in captivity such as a bear, a panda, a lion, a tiger, a leopard, an elephant, a zebra, a giraffe, a gorilla, a dolphin, or a whale. In some embodiments, subjects are “patients,” i.e., living humans or animals that are receiving medical care for a disease or condition, which includes persons or animals with no defined illness who are being evaluated for signs of pathology. In some embodiments, subjects who can be treated using the methods include subjects identified or selected as having or being at risk for having cancer. Such identification and/or selection can be made by clinical or diagnostic evaluation.

Specific values listed herein for radicals, substituents, and ranges are for illustration purposes only unless otherwise specified; such examples do not exclude other defined values or other values within defined ranges for the radicals and substituents. For example, (C₁-C₆)alkyl can be methyl, ethyl, propyl, isopropyl, butyl, iso-butyl, sec-butyl, pentyl, 3-pentyl, or hexyl; (C₁-C₃)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; (C₁-C₃)alkoxy can be methoxy, ethoxy, or propoxy; and (C₂-C₆)alkanoyloxy can be acetoxy, propanoyloxy, butanoyloxy, isobutanoyloxy, pentanoyloxy, or hexanoyloxy.

Where substituent groups are specified by the conventional chemical formulae, written from left to right, they equally encompass the chemically identical substituents that would result from writing the structure from right to left, e.g., —CH₂O— is equivalent to —OCH₂—.

Alkyl, alkoxy, etc. denote a straight (i.e., unbranched) or branched chain, or a combination thereof, which may be fully saturated, mono- or polyunsaturated and can include di- and multivalent radicals, having the number of carbon (C) atoms designated (i.e., C₁-C₁₀ means one to ten carbons). Examples of saturated hydrocarbon radicals include, without limitation, groups such as methyl, ethyl, n-propyl, isopropyl, n-butyl, t-butyl, isobutyl, sec-butyl, and (cyclohexyl)methyl, and homologs and isomers of, for example, n-pentyl, n-hexyl, n-heptyl, n-octyl, and the like. An unsaturated alkyl group is one having one or more double bonds or triple bonds. Examples of unsaturated alkyl groups include, without limitation, vinyl, 2-propenyl, crotyl-2-isopentenyl, 2-(butadienyl), 2,4-pentadienyl, 3-(1,4-penadienyl), ethynyl, 1- and 3-propynyl, 3-butynyl, and the higher homologs and isomers. An alkoxy is an alkyl attached to the remainder of the molecule via an oxygen linker (—O—). In some embodiments, alkoxy refers to a radical bonded through an oxygen atom of the formula —O-alkyl.

In general, the term “acyl” or “acyl substituent” refers to a substituent derived by the removal of one or more hydroxyl groups from an oxoacid, including inorganic acids, and contains a double-bonded oxygen atom and an alkyl group. Further, reference to an individual radical, such as “propyl,” embraces only the straight chain radical; a branched chain isomer, such as “isopropyl,” is specifically referenced.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass, all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

1. A method of treating a cancer in a subject, which method comprises administering to the subject a therapeutically effective amount of:
a small molecule drug conjugate (SMDC) or a pharmaceutically acceptable salt thereof comprising:
(a) a drug moiety selected from the group consisting of a phosphoinositide 3-kinase (PI3K) inhibitor, a stimulator of interferon genes (STING) agonist, nucleotide-binding oligomerization domain (NOD)-like receptor (NLR) agonist, a retinoic acid-inducible gene-I (RIG-I)-like receptor (RLR) agonist, an absent in melanoma 2 (AIM2)-like receptor (ALR) agonist, a receptor for advanced glycation end products (RAGE) agonist, a kinase of the Pelle/interleukin-1 (IL-1) receptor-associated kinase 1 (IRAK1) agonist, an IRAK2 agonist, an IRAK4 agonist, an IRAK3 antagonist, an Src homology 2 domain-containing tyrosine phosphatase 1 and 2 (SHP1/2) antagonist, a T cell protein tyrosine phosphatase (TC-PTP) antagonist, a diacylglycerol kinase (DGK) antagonist, an enhancer of zeste homolog 2 (EZH2) antagonist, a transforming growth factor beta (TGFβ) antagonist, a nuclear factor kappa-light-chain-enhancer of activated B cells (NFκβ) activator, or a 1-kappa-β (Iκβ) kinase antagonist; and
(b) a ligand selected from the group consisting of a radical of raltitrexed, a radical of 5-methyltetrahydrofolate, or a group comprising formula (X): 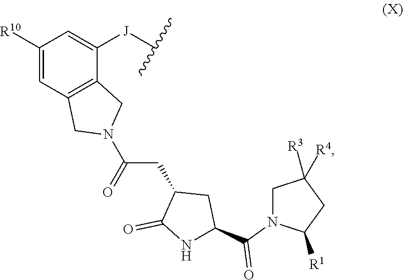
wherein:
J is (C(R^{J})₂)₀₋₃, wherein each R^{J} is H, or two or more R^{J} are taken together to form oxo;
R¹ is selected from the group consisting of —CN, —CHO, and —B(OH)₂;
R³ and R⁴ are each independently selected from the group consisting of —H and F; and
R¹⁰ is selected from the group consisting of H, —CF₃, F, Cl, Br and I; and
(c) a linker connecting the ligand and the drug moiety; and
a chimeric antigen receptor (CAR)-expressing cytotoxic lymphocyte.

2-5. (canceled)

6. The method of claim 1, wherein the ligand comprises formula (XA), formula (XB), formula (XC), or formula (XD):

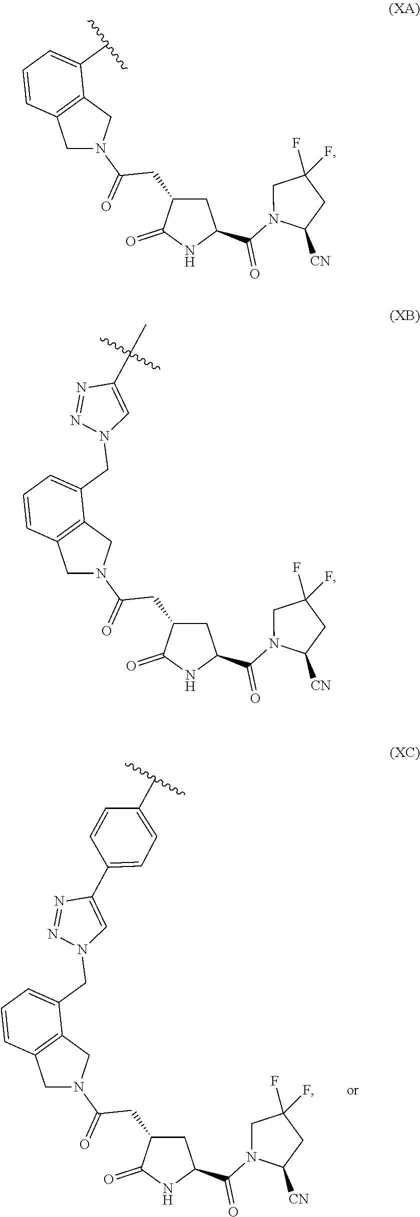 [SMILES: *c1cccc2c1CN(C(=O)C[C@@H]1C[C@@H](C(=O)N3CC(F)(F)C[C@H]3C#N)NC1=O)C2 ; *c1cn(Cc2cccc3c2CN(C(=O)C[C@@H]2C[C@@H](C(=O)N4CC(F)(F)C[C@H]4C#N)NC2=O)C3)nn1 ; *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

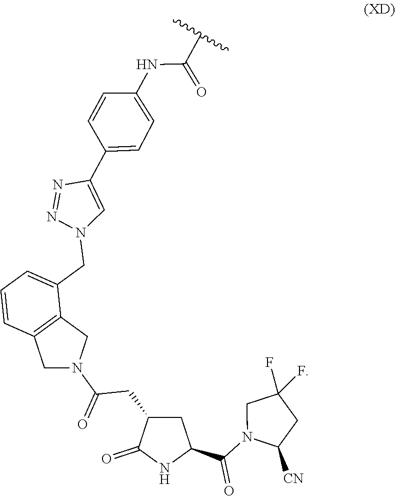 [SMILES: *C(=O)Nc1ccc(-c2cn(Cc3cccc4c3CN(C(=O)C[C@@H]3C[C@@H](C(=O)N5CC(F)(F)C[C@H]5C#N)NC3=O)C4)nn2)cc1]

7-45. (canceled)

46. The method of claim 1, wherein the linker comprises a releasable form of polyethylene glycol (PEG), a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of polyethylene glycol.

47. The method of claim 1, wherein the linker is a releasable linker.

48. The method of claim 1, wherein the linker is a non-releasable linker.

49. The method of claim 1, wherein the linker comprises a group represented by the structure: 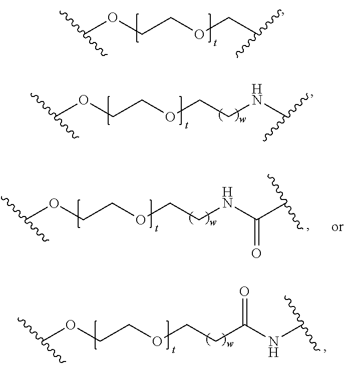
wherein w is 0-5 and t is 1-30, or the linker comprises one or more linker groups having the following structure: 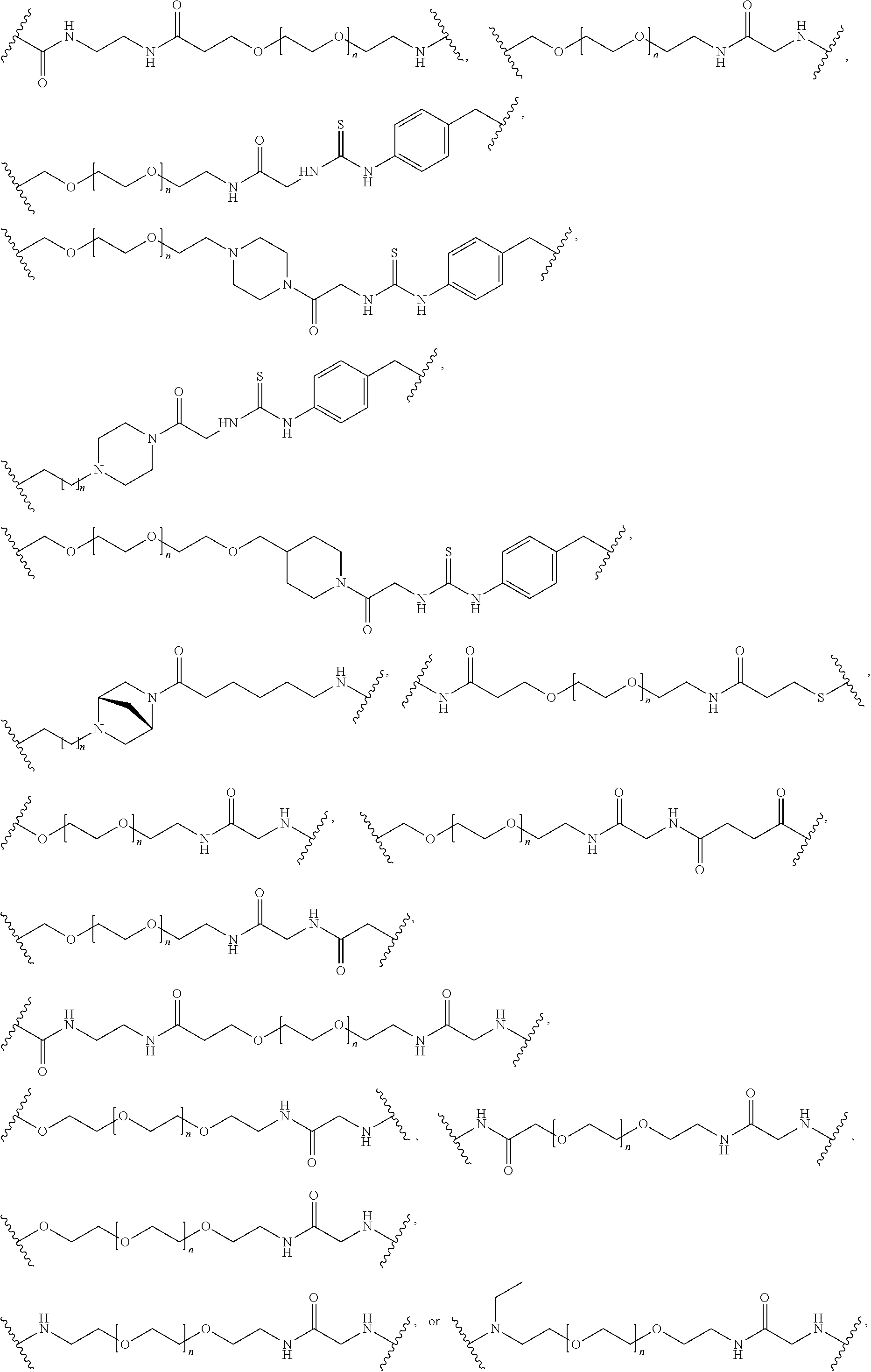
wherein n is 0 to 15.

50. (canceled)

51. The method of claim 1, wherein the SMDC further comprises a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker.

52. (canceled)

53. The method of claim 1, wherein each CAR comprises a recognition region, a co-stimulation domain, and an activation signaling domain, wherein the recognition region of the CAR is a single chain variable fragment (scFV) of an antibody that binds to a cell-surface antigen on an immunosuppressive cell or a cancer cell with high specificity.

54-56. (canceled)

57. The method of claim 53, wherein the cell-surface antigen is a tumor-associated antigen (TAA).

58. The method of claim 53, wherein the CAR-expressing cytotoxic lymphocyte is a T cell and the TAA is selected from the group consisting of CD19, CD20, CD30, CD3, CD4, CD5, BCMA, GD2, LEY, MS4A1, CD22, TNFRSF17, CD38, SDC1, TNFRSF8, IL3RA, CD7, NCAM1, CD34, CLEC12A, CD4, MME, CD5, SLAMF7, ILIRAP, FCGR3A, ITGB7, TNFRSF13B,TRBC1, CD33, ROR1, MUC1, KLRK1, KIT, CD274, CD70, PROM1, AFP,AXL, CD80, CD86, DLL3, TNFRSF10B, FAP, MAGEA1, MAGEA4, MUC16, PMEL, ROR2, KDR, EPHA2, L1CAM, CLDN18, PSCA, FOLR1, IL13RA2, MET, EPCAM, EGFR, EGFRVIII, FOLH1, GPC3, CEACAM5, ERBB2, CAIX, B4GALNT1, NY-ESO-1, PD-L1, and MSLN.

59. The method of claim 57, wherein the TAA is CD19.

60. The method of claim 53, wherein the CAR-expressing cytotoxic lymphocyte is a T cell and the co-stimulation domain of the CAR is CD27, CD40L, CD70, 2B4, DNAM1, DAP12, DAP10, NKG2, NKG2D, CD28, CD137 (4-1BB), CD134 (OX40), or CD278 (ICOS).

61. The method of claim 53, wherein the CAR-expressing cytotoxic lymphocyte is a NK cell and the TAA is selected from the group consisting of CD3, CD4, and CD5.

62. The method of claim 53, wherein the CAR-expressing cytotoxic lymphocyte is a NK cell and the co-stimulation domain of the CAR is selected from the group consisting of 2B4, CD137 (4-1BB), DNAM1, DAP12, DAP10, NKG2, and NKG2D.

63. (canceled)

64. The method of claim 1, wherein the cytotoxic lymphocyte is autologous or heterolgous to the individual.

65-69. (canceled)

70. The method of claim 1, wherein the ligand is a radical of raltitrexed or a radical of 5-methyltetrahydrofolate and the cancer is a folate receptor-expressing cancer or a cancer mediated by folate receptor-expressing immune cells.

71. The method of claim 70, wherein the cancer is a folate receptor α-expressing cancer, a folate receptor β-expressing cancer, a folate receptor δ-expressing cancer, or a cancer mediated by folate receptor α, β, or δ-expressing immune cells.

72. The method of claim 1, wherein the ligand is a group of formula (X), and the cancer is a cancer expressing fibroblast activation protein (FAP) or a cancer with cancer associated fibroblasts which express FAP.

73. The method of claim 1, wherein administration of the therapeutically effective amount of the SMDC or a pharmaceutically acceptable salt thereof inhibits cancer-associated fibroblast activity in the subject and increases the efficacy of the administered CAR-expressing cytotoxic lymphocytes against the cancer as compared to the efficacy of the CAR-expressing cytotoxic lymphocytes administered in the absence of the administration of a therapeutically effective amount of the SMDC or a pharmaceutically acceptable salt thereof.

74-101. (canceled)

102. A synergistic combination for treating a solid tumor cancer comprising:
(i) a therapeutically effective amount of a small molecule drug conjugate (SMDC) or pharmaceutically acceptable salt thereof comprising:
(a) a drug moiety selected from the group consisting of a phosphoinositide 3-kinase (PI3K) inhibitor, a stimulator of interferon genes (STING) agonist, nucleotide-binding oligomerization domain (NOD)-like receptor (NLR) agonist, a retinoic acid-inducible gene-I (RIG-I)-like receptor (RLR) agonist, an absent in melanoma 2 (AIM2)-like receptor (ALR) agonist, a receptor for advanced glycation end products (RAGE) agonist, a kinase of the Pelle/interleukin-1 (IL-1) receptor-associated kinase 1 (IRAK1) agonist, an IRAK2 agonist, an IRAK4 agonist, an IRAK3 antagonist, an Src homology 2 domain-containing tyrosine phosphatase 1 and 2 (SHP1/2) antagonist, a T cell protein tyrosine phosphatase (TC-PTP) antagonist, a diacylglycerol kinase (DGK) antagonist, an enhancer of zeste homolog 2 (EZH2) antagonist, a transforming growth factor beta (TGFβ) antagonist, a nuclear factor kappa-light-chain-enhancer of activated B cells (NFκβ) activator, or a 1-kappa-β (Iκβ) kinase antagonist;
(b) a ligand selected from the group consisting of a radical of raltitrexed, a radical of 5-methyltetrahydrofolate, or a group comprising formula (X): 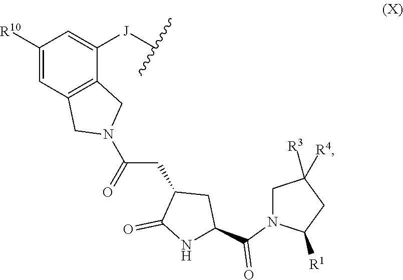
wherein:
J is (C(R^{J})₂)₀₋₃, wherein each R^{J} is H, or two or more R^{J} are taken together to form oxo;
R¹ is selected from the group consisting of —CN, —CHO, and —B(OH)₂;
R³ and R⁴ are each independently selected from the group consisting of —H and F; and
R¹⁰ is selected from the group consisting of H, —CF₃, F, Cl, Br and I; and
(c) a linker connecting the ligand and the drug moiety; and
(ii) a therapeutically effective amount of a CAR-expressing cytotoxic lymphocyte.
